(19) United States
(12) Patent Application Publication
(10) Pub. No.: US 2019/0247299 A1
(43) Pub. Date: Aug. 15, 2019

(54) COMPOSITIONS AND METHODS FOR TREATING ECZEMA

(71) Applicant: C&A Innovations Corp., Denver, CO (US)

(72) Inventors: Michael CAMERON, Denver, CO (US); James ALLRED, Alvaton, KY (US)

(21) Appl. No.: 16/275,550

(22) Filed: Feb. 14, 2019

Related U.S. Application Data
(60) Provisional application No. 62/717,298, filed on Aug. 10, 2018.
(60) Provisional application No. 62/631,045, filed on Feb. 15, 2018.

(51) Int. Cl. A61K 9/00; A61K 36/28; A61K 36/889; A61K 36/185; A61K 36/82; A61K 31/01; A61K 31/355; A61K 31/045; A61K 31/05

(52) U.S. Cl. CPC A61K 9/0014; A61K 36/28; A61K 36/889; A61K 36/185; A61K 31/05; A61K 31/01; A61K 31/355; A61K 31/045; A61K 36/82

(57) ABSTRACT

Topical compositions comprising sunflower oil, coconut oil, shea butter, grapeseed oil, squalene, and a tocopherol are disclosed. Topical compositions comprising sunflower oil, coconut oil, shea butter, camellia oil, squalane, and a tocopherol are further disclosed. Topical compositions comprising sunflower oil, coconut oil, shea butter, camellia oil, squalane, menthol and a tocopherol are disclosed. Topical compositions comprising sunflower oil, coconut oil, shea butter, camellia oil, squalane, hemp oil and a tocopherol are also disclosed. Topical compositions comprising sunflower oil, coconut oil, shea butter, camellia oil, squalane, menthol, hemp oil and a tocopherol are also disclosed. The compositions disclosed herein can be used for improving various skin conditions, such as eczema, erythema, irritation, pruritus, drying, drying of the skin, cracking of the skin, wrinkles, rosacea, contact dermatitis, and atopic dermatitis.

### CLAIM OF PRIORITY

This application claims priority to U.S. provisional Application No. 62/631,045, filed on Feb. 15, 2018, entitled “Compositions and Methods for Treating Eczema,” and U.S. Provisional Application No. 62/717,298, filed on Aug. 10, 2018, entitled “Compositions and Methods for Treating Eczema,” and each of these are incorporated herein by reference.

### SUMMARY

Embodiments of the invention are directed to compositions comprising sunflower oil, coconut oil, shea butter, grapeseed oil, squalene, and a tocopherol.

Embodiments of the invention are directed to compositions comprising sunflower oil, coconut oil, shea butter, camellia oil, squalane, cetyl alcohol, and a tocopherol.

Embodiments of the invention are directed to compositions comprising sunflower oil, coconut oil, shea butter, camellia oil, squalane, cetyl alcohol, menthol, and a tocopherol.

Embodiments of the invention are directed to compositions comprising sunflower oil, coconut oil, shea butter, camellia oil, squalane, cetyl alcohol, hemp oil, and a tocopherol.

Embodiments of the invention are directed to compositions comprising sunflower oil, coconut oil, shea butter, camellia oil, squalane, cetyl alcohol, cannabidiol, and a tocopherol.

Embodiments of the invention are directed to compositions comprising sunflower oil, coconut oil, shea butter, camellia oil, squalane, cetyl alcohol, menthol, hemp oil, and a tocopherol.

Embodiments of the invention are directed to compositions comprising sunflower oil, coconut oil, shea butter, camellia oil, squalane, cetyl alcohol, menthol, cannabidiol, and a tocopherol.

Embodiments of the invention are directed to compositions comprising sunflower oil, coconut oil, shea butter, camellia oil, squalane or squalene, cetyl alcohol, menthol, hemp oil, cannabidiol, and a tocopherol.

Embodiments of the invention are directed to methods of ameliorating a skin condition in a subject in need thereof comprising topically administering to the subject a composition comprising sunflower oil, coconut oil, shea butter, grapeseed oil, squalene, and a tocopherol.

Embodiments of the invention are directed to methods of ameliorating a skin condition in a subject in need thereof comprising topically administering to the subject a composition comprising sunflower oil, coconut oil, shea butter, camellia oil, squalane, cetyl alcohol, and a tocopherol.

Embodiments of the invention are directed to methods of ameliorating a skin condition in a subject in need thereof comprising topically administering to the subject a composition comprising sunflower oil, coconut oil, shea butter, camellia oil, squalane, cetyl alcohol, menthol, and a tocopherol.

Embodiments of the invention are directed to methods of ameliorating a skin condition in a subject in need thereof comprising topically administering to the subject a composition comprising sunflower oil, coconut oil, shea butter, camellia oil, squalane, cetyl alcohol, hemp oil, and a tocopherol.

Embodiments of the invention are directed to methods of ameliorating a skin condition in a subject in need thereof comprising topically administering to the subject a composition comprising sunflower oil, coconut oil, shea butter, camellia oil, squalane, cetyl alcohol, cannabidiol, and a tocopherol.

Embodiments of the invention are directed to methods of ameliorating a skin condition in a subject in need thereof comprising topically administering to the subject a composition comprising sunflower oil, coconut oil, shea butter, camellia oil, squalane, cetyl alcohol, menthol, hemp oil, and a tocopherol.

Embodiments of the invention are directed to methods of ameliorating a skin condition in a subject in need thereof comprising topically administering to the subject a composition comprising sunflower oil, coconut oil, shea butter, camellia oil, squalane, cetyl alcohol, menthol, cannabidiol, and a tocopherol.

Embodiments of the invention are directed to methods of ameliorating a skin condition in a subject in need thereof comprising topically administering to the subject a composition comprising sunflower oil, coconut oil, shea butter, camellia oil, squalane, cetyl alcohol, menthol, hemp oil, cannabidiol, and a tocopherol.

Embodiments of the invention are directed to methods of treating eczema in a subject in need thereof comprising topically administering to the subject a composition comprising sunflower oil, coconut oil, shea butter, grapeseed oil, squalene, and a tocopherol.

Embodiments of the invention are directed to methods of treating eczema in a subject in need thereof comprising topically administering to the subject a composition comprising sunflower oil, coconut oil, shea butter, camellia oil, squalane, cetyl alcohol, and a tocopherol.

Embodiments of the invention are directed to methods of treating eczema in a subject in need thereof comprising topically administering to the subject a composition comprising sunflower oil, coconut oil, shea butter, camellia oil, squalane, cetyl alcohol, menthol, and a tocopherol.

Embodiments of the invention are directed to methods of treating eczema in a subject in need thereof comprising topically administering to the subject a composition comprising sunflower oil, coconut oil, shea butter, camellia oil, squalane, cetyl alcohol, hemp oil, and a tocopherol.

Embodiments of the invention are directed to methods of treating eczema in a subject in need thereof comprising topically administering to the subject a composition comprising sunflower oil, coconut oil, shea butter, camellia oil, squalane, cetyl alcohol, cannabidiol, and a tocopherol.

Embodiments of the invention are directed to methods of treating eczema in a subject in need thereof comprising topically administering to the subject a composition comprising sunflower oil, coconut oil, shea butter, camellia oil, squalane, cetyl alcohol, menthol, hemp oil, and a tocopherol.

Embodiments of the invention are directed to methods of treating eczema in a subject in need thereof comprising topically administering to the subject a composition comprising sunflower oil, coconut oil, shea butter, camellia oil, squalane, cetyl alcohol, menthol, cannabidiol, and a tocopherol.

Embodiments of the invention are directed to methods of treating eczema in a subject in need thereof comprising topically administering to the subject a composition comprising sunflower oil, coconut oil, shea butter, camellia oil, squalane, cetyl alcohol, menthol, hemp oil, cannabidiol, and a tocopherol.

Embodiments of the invention are directed to methods of improving skin texture or appearance in a subject in need thereof comprising topically administering to the subject a composition comprising sunflower oil, coconut oil, shea butter, grapeseed oil, squalene, and a tocopherol.

Embodiments of the invention are directed to methods of improving skin texture or appearance in a subject in need thereof comprising topically administering to the subject a composition comprising sunflower oil, coconut oil, shea butter, camellia oil, squalane, cetyl alcohol, and a tocopherol.

Embodiments of the invention are directed to methods of improving skin texture or appearance in a subject in need thereof comprising topically administering to the subject a composition comprising sunflower oil, coconut oil, shea butter, camellia oil, squalane, cetyl alcohol, menthol, and a tocopherol.

Embodiments of the invention are directed to methods of improving skin texture or appearance in a subject in need thereof comprising topically administering to the subject a composition comprising sunflower oil, coconut oil, shea butter, camellia oil, squalane, cetyl alcohol, hemp oil and a tocopherol.

Embodiments of the invention are directed to methods of improving skin texture or appearance in a subject in need thereof comprising topically administering to the subject a composition comprising sunflower oil, coconut oil, shea butter, camellia oil, squalane, cetyl alcohol, cannabidiol, and a tocopherol.

Embodiments of the invention are directed to methods of improving skin texture or appearance in a subject in need thereof comprising topically administering to the subject a composition comprising sunflower oil, coconut oil, shea butter, camellia oil, squalane, cetyl alcohol, menthol, hemp oil, and a tocopherol.

Embodiments of the invention are directed to methods of improving skin texture or appearance in a subject in need thereof comprising topically administering to the subject a composition comprising sunflower oil, coconut oil, shea butter, camellia oil, squalane, cetyl alcohol, menthol, cannabidiol, and a tocopherol.

Embodiments of the invention are directed to methods of improving skin texture or appearance in a subject in need thereof comprising topically administering to the subject a composition comprising sunflower oil, coconut oil, shea butter, camellia oil, squalane, cetyl alcohol, menthol, hemp oil, cannabidiol, and a tocopherol.

Embodiments of the invention are directed to methods of improving the appearance of or treating eczema in a subject in need thereof comprising topically administering to the subject a morning regimen composition comprising sunflower oil, coconut oil, shea butter, grapeseed oil, squalene, and a tocopherol, and an evening regimen composition comprising sunflower oil, coconut oil, shea butter, grapeseed oil, squalene, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, and a tocopherol, an evening regimen composition comprising sunflower oil, coconut oil, shea butter, camellia oil, squalane, cetyl alcohol, and a tocopherol, an after wash regimen composition comprising sunflower oil, coconut oil, shea butter, camellia oil, squalene, cetyl alcohol, and a tocopherol, and an anti-itch regimen composition comprising sunflower oil, coconut oil, shea butter, camellia oil, squalane, cetyl alcohol, menthol, and a tocopherol.

Embodiments of the invention are directed to methods of improving the appearance of or treating eczema in a subject in need thereof comprising topically administering to the subject an anti-itch regimen composition comprising sunflower oil, coconut oil, shea butter, camellia oil, squalane, cetyl alcohol, hemp oil, and a tocopherol.

Embodiments of the invention are directed to methods of improving the appearance of or treating eczema in a subject in need thereof comprising topically administering to the subject an anti-itch regimen composition comprising sunflower oil, coconut oil, shea butter, camellia oil, squalane, cetyl alcohol, cannabidiol, and a tocopherol.

Embodiments of the invention are directed to methods of improving the appearance of or treating eczema in a subject in need thereof comprising topically administering to the subject an anti-itch regimen composition comprising sunflower oil, coconut oil, shea butter, camellia oil, squalane, cetyl alcohol, menthol, hemp oil, and a tocopherol.

Embodiments of the invention are directed to methods of improving the appearance of or treating eczema in a subject in need thereof comprising topically administering to the subject an anti-itch regimen composition comprising sunflower oil, coconut oil, shea butter, camellia oil, squalane, cetyl alcohol, menthol, cannabidiol, and a tocopherol.

Embodiments of the invention are directed to methods of improving the appearance of or treating eczema in a subject in need thereof comprising topically administering to the subject an anti-itch regimen composition comprising sunflower oil, coconut oil, shea butter, camellia oil, squalane, cetyl alcohol, menthol, hemp oil, cannabidiol, and a tocopherol.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 and 7 μm are also explicitly disclosed, as well as the range of values greater than or equal to 1 μm and the range of values less than or equal to 8 μm.

All percentages, parts and ratios are based upon the total weight of the topical compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g, “about 50” means 45 to 55, “about 25,000” means 22,500 to 27,500, etc, unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g, more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “topical” includes administering to any skin or mucosal surface. Skin surface includes any part of the body, including but not limited to face, hands, legs, neck, abdominal area, eyes, nose, and chest. Mucosal surface includes, without limitation, mucosa of the mouth, lips, tongue, buccal mucosa, palate, gingiva, nasopharynx, respiratory epithelium, conjunctiva, vagina, cervix, and urethral mucosa.

The term “treating” is used herein, for instance, in reference to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

The term “wt. % (weight percent)” as used herein may be weight-to-weight or weight-to-volume percentages with respect to the total amount of the composition, as applicable.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

### Compositions

Embodiments of the invention are directed to topical compositions comprising sunflower oil, coconut oil, shea butter, grapeseed oil, squalene, and a tocopherol.

Embodiments of the invention are directed to topical compositions comprising sunflower oil, coconut oil, shea butter, camellia oil, squalane, cetyl alcohol, and a tocopherol.

Embodiments of the invention are directed to topical compositions comprising sunflower oil, coconut oil, shea butter, camellia oil, squalane, cetyl alcohol, menthol, and a tocopherol.

Embodiments of the invention are directed to topical compositions comprising sunflower oil, coconut oil, shea butter, camellia oil, squalane, cetyl alcohol, hemp oil, and a tocopherol.

Embodiments of the invention are directed to topical compositions comprising sunflower oil, coconut oil, shea butter, camellia oil, squalane, cetyl alcohol, cannabidiol, and a tocopherol.

Embodiments of the invention are directed to topical compositions comprising sunflower oil, coconut oil, shea butter, camellia oil, squalane, cetyl alcohol, menthol, hemp oil, and a tocopherol.

Embodiments of the invention are directed to topical compositions comprising sunflower oil, coconut oil, shea butter, camellia oil, squalane, cetyl alcohol, menthol, cannabidiol, and a tocopherol.

In embodiments, the sunflower oil may be present from about 2 wt. % to about 20 wt. % of the composition, about 2 wt. % to about 15 wt. % of the composition, about 2 wt. % to about 10 wt. % of the composition, about 2 wt. % to about 8 wt. % of the composition, about 2 wt. % to about 6 wt. % of the composition, or about 2 wt. % to about 4 wt. % of the composition. Specific examples include about 2 wt. %, about 4 wt. %, about 5 wt. %, about 6 wt. %, about 7 wt. %, about 8 wt. %, about 10 wt. %, about 12 wt. %, about 15 wt. %, about 20 wt. %, and any individual amount or any ranges between any two of these values. Sunflower oil naturally has a fatty acid profile consisting of palmitic acid (4-8% of the total fatty acids), stearic acid (2-6% of the total fatty acids), oleic acid (20-45% of the total fatty acids) and linoleic acid (45-70% of the total fatty acids). The sunflower oil can be from any source, including extracts of sunflower seeds, and other commercially available hybrid sunflower seeds having high stearic and oleate content.

In embodiments, the coconut oil may be present from about 2 wt. % to about 20 wt. % of the composition, about 2 wt. % to about 15 wt. % of the composition, about 2 wt. % to about 10 wt. % of the composition, about 2 wt. % to about 8 wt. % of the composition, about 2 wt. % to about 6 wt. % of the composition, about 2 wt. % to about 5 wt. % of the composition, or about 2 wt. % to about 4 wt. % of the composition. Specific examples include about 2 wt. %, about 4 wt. %, about 5 wt. % about 6 wt. %, about 10 wt. %, about 12 wt. %, about 15 wt. %, about 20 wt. %, and any individual amount or any ranges between any two of these values. Coconut oil can be from any source, and typically extracted directly from coconut meat and separated from the water content, and is called virgin or unrefined coconut oil. This virgin or unrefined coconut oil has a melting point that may range from 76° F. to 92° F. and is also known generally as “coconut oil 76” and “coconut oil 92.” In embodiments, fractionated coconut oil can also be used, and refers to coconut oil that has been refined, for example, to comprise only the saturated fat portion of the oil by removing the long-chain triglycerides.

In embodiments, the shea butter may be present from about 2 wt. % to about 20 wt. % of the composition, about 2 wt. % to about 15 wt. % of the composition, about 2 wt. % to about 10 wt. % of the composition, about 2 wt. % to about 8 wt. % of the composition, about 2 wt. % to about 6 wt. % of the composition, or about 2 wt. % to about 4 wt. % of the composition. Specific examples include about 2 wt. %, about 3 wt. %, about 4 wt. %, about 5 wt. %, about 10 wt. %, about 12 wt. %, about 15 wt. %, about 20 wt. %, and any individual amount or any ranges between any two of these values. Shea butter generally has a fatty acid profile of oleic acid (40-60%), stearic acid (20-50%), linoleic acid (3-11%), palmitic acid (2-9%), linolenic acid (<1%) and arachidic acid (<1%). The compositions disclosed herein may contain refined or unrefined shea butter.

In embodiments, the grape seed oil may be present from about 0.1 wt. % to about 10 wt. % of the composition, about 0.1 wt. % to about 8 wt. % of the composition, about 0.1 wt. % to about 6 wt. % of the composition, about 0.1 wt. % to about 4 wt. % of the composition, about 0.1 wt. % to about 2 wt. % of the composition, or about 0.1 wt. % to about 1 wt. % of the composition. Specific examples include about 0.1 wt. %, about 2 wt. %, about 3 wt. %, about 5 wt. %, about 8 wt. %, about 9 wt. %, about 10 wt. %, and any individual amount or any ranges between any two of these values. Grape seed oil usually contain cyclic polyphenols, such as procyanidines (epigallocatechin-3-gallates), catechin, epicatechin, procyanidines B1, B2 and B3, procyanidine C1, and various proanthocyanidines P1-P2. Grape seed oil can be obtained from any source, such as from crushed grape seeds or from cold-pressed grape seeds.

In embodiments, squalene may be present from about 0.1 wt. % to about 10 wt. % of the composition, about 0.1 wt. % to about 8 wt. % of the composition, about 0.1 wt. % to about 6 wt. % of the composition, about 0.1 wt. % to about 4 wt. % of the composition, about 0.1 wt. % to about 2 wt. % of the composition, or about 0.1 wt. % to about 1 wt. % of the composition. Specific examples include about 0.1 wt. %, about 2 wt. %, about 3 wt. %, about 5 wt. %, about 8 wt. %, about 9 wt. %, about 10 wt. %, and any individual amount or any ranges between any two of these values. Squalene can be from any source, such as fish, shark, plants, and genetically engineered microorganisms. Squalene can be from a lipid bilayer from any source, such as fish, shark, plants, and genetically engineered microorganisms.

In embodiments, squalane may be present from about 0.1 wt. % to about 10 wt. % of the composition, about 0.1 wt. % to about 8 wt. % of the composition, about 0.1 wt. % to about 6 wt. % of the composition, about 0.1 wt. % to about 4 wt. % of the composition, about 0.1 wt. % to about 2 wt. % of the composition, or about 0.1 wt. % to about 1 wt. % of the composition. Specific examples include about 0.1 wt. %, about 2 wt. %, about 3 wt. %, about 5 wt. %, about 6 wt. %, about 8 wt. %, about 9 wt. %, about 10 wt. %, and any individual amount or any ranges between any two of these values. Squalane can be from any source, such as fish, shark, plants, and genetically engineered microorganisms. Squalane can be from a lipid bilayer from any source, such as fish, shark, plants, and genetically engineered microorganisms. In embodiments, squalane can be from sebaceous secretions. In embodiments, squalane can be derived by hydrogenation of squalene. In embodiments, squalane is not auto-oxidized. In embodiments, and as used and described in the compositions herein, squalene and squalane can be used interchangeably.

In embodiments, the camellia oil may be present from about 2 wt. % to about 20 wt. % of the composition, about 2 wt. % to about 15 wt. % of the composition, about 2 wt. % to about 10 wt. % of the composition, about 2 wt. % to about 9 wt. % of the composition, about 2 wt. % to about 7 wt. % of the composition, or about 2 wt. % to about 4 wt. % of the composition. Specific examples include about 2 wt. %, about 4 wt. %, about 5 wt. %, about 6 wt. %, about 7 wt. %, about 8 wt. %, about 9 wt. %, about 10 wt. %, about 12 wt. %, about 15 wt. %, about 20 wt. %, and any individual amount or any ranges between any two of these values. The camellia oil can be from any source, including extracts of camellia seeds, camellia oleifera seeds, and other commercially available hybrid camellia seeds.

In embodiments, the menthol may be present from about 0.1 wt. % to about 10 wt. % of the composition, about 0.1 wt. % to about 5 wt. % of the composition, about 0.1 wt. % to about 2.5 wt. % of the composition, about 0.1 wt. % to about 1 wt. % of the composition, or about 0.1 wt. % to about 0.5 wt. % of the composition. Specific examples include about 0.1 wt. %, about 0.2 wt. %, about 0.4 wt. %, about 0.6 wt. %, about 0.8 wt. %, about 1 wt. %, about 2.5 wt. %, about 5 wt. %, about 10 wt. %, and any individual amount or any ranges between any two of these values.

In embodiments, the hemp oil may be present from about 0.1 wt. % to about 20 wt. % of the composition, about 0.1 wt. % to about 15 wt. % of the composition, about 0.1 wt. % to about 10 wt. % of the composition, about 0.1 wt. % to about 8 wt. % of the composition, about 0.1 wt. % to about 6 wt. % of the composition, about 0.1 wt. % to about 4 wt. % of the composition, about 0.1 wt. % to about 2 wt. % of the composition, about 0.1 wt. % to about 1 wt. % of the composition, about 0.1 wt. % to about 0.5 wt. % of the composition, or about 0.1 wt. % to about 0.25 wt. % of the composition. Specific examples include about 0.1 wt. %, 0.25 wt. %, 0.5 wt. %, 1 wt. %, 2 wt. %, about 4 wt. %, about 5 wt. %, about 6 wt. %, about 7 wt. %, about 8 wt. %, about 10 wt. %, about 12 wt. %, about 15 wt. %, about 20 wt. %, and any individual amount or any ranges between any two of these values. The hemp oil can be from any source, including extracts of hemp seeds, and other commercially available hybrid hemp seeds. The hemp oil also includes full spectrum hemp oil.

In embodiments, the cannabidiol may be present from about 0.1 wt. % to about 20 wt. % of the composition, about 0.1 wt. % to about 15 wt. % of the composition, about 0.1 wt. % to about 10 wt. % of the composition, about 0.1 wt. % to about 8 wt. % of the composition, about 0.1 wt. % to about 6 wt. % of the composition, about 0.1 wt. % to about 4 wt. % of the composition, about 0.1 wt. % to about 2 wt. % of the composition, about 0.1 wt. % to about 1 wt. % of the composition, about 0.1 wt. % to about 0.5 wt. % of the composition, or about 0.1 wt. % to about 0.25 wt. % of the composition. Specific examples include about 0.1 wt. %, 0.25 wt. %, 0.5 wt. %, 1 wt. %, 2 wt. %, about 4 wt. %, about 5 wt. %, about 6 wt. %, about 7 wt. %, about 8 wt. %, about 10 wt. %, about 12 wt. %, about 15 wt. %, about 20 wt. %, and any individual amount or any ranges between any two of these values. The cannabidiol can be from any source, including extracts of cannabis seeds, and other commercially available cannabis seeds. In some embodiments, the composition may include other cannabinoids, such as THC (tetrahydrocannabinol), THCA (tetrahydrocannabinolic acid), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), CBT (cannabicitran), beta-caryophyllene, 2-AG (2-arachidonoylglycerol), AEA (Anandamide), N-arachadonoyl dopamine, HEA (homolineoleoyl ethanolamide), DEA (docosa tetraenyl ethanolamide), virodhamine, noladin ether, PEA (palmitoyl ethanolamide), OEA (Oleoylethanolamide), WIN-55,212-2, JWH-133, MET (R-methanandamide), and CP 55,940. The cannabinoids may be present in the composition in the ranges disclosed herein.

In embodiments, tocopherol may be present from about 0.1 wt. % to about 5 wt. % of the composition, about 0.1 wt. % to about 4 wt. % of the composition, about 0.1 wt. % to about 3 wt. % of the composition, about 0.1 wt. % to about 2 wt. % of the composition, about 0.1 wt. % to about 1 wt. % of the composition, or about 0.1 wt. % to about 0.5 wt. % of the composition. Specific examples include about 0.1 wt. %, about 1 wt. %, about 1.5 wt. %, about 2 wt. %, about 3 wt. %, about 4 wt. %, about 5 wt. %, and any individual amount or any ranges between any two of these values. Tocopherol includes alpha-tocopherol, beta-tocopherol, gamma-tocopherol, delta-tocopherols, and mixtures thereof (tocopherol-50), tocopherol esters, tocopherol sorbate, and the like.

Other non-limiting examples of natural oils and plant oils that may be used in the compositions are abyssinian oil, almond oil, apricot kernel oil, argan nut oil, avocado oil, babassu oil, baobab oil, black cumin oil, black currant oil, borage oil, brazil nut oil, camelina oil, canola oil, castor oil, cherry kernel oil, chai seed oil, corn oil, cranberry seed oil, echium oil, evening primrose oil, eucalyptus oil, flax seed oil, grapefruit oil, hazelnut oil, hemp seed oil, jojoba oil, Kalahari melon oil, kukui nut oil, linseed oil, macadamia nut oil, marula oil, meadowfoam seed oil, monoi oil, moringa oil, neem oil, oat oil, olive oil, palm fruit oil, palm kernel oil, passion fruit seed oil, peach kernel oil, pecan oil, perilla seed oil, peanut oil, pistachio oil, pomegranate seed oil, prickly pear seed oil, pumpkin seed oil, raspberry seed oil, rice bran oil, rosehip oil, sacha inchi oil, safflower oil, seabuckthorn fruit oil, sesame seed oil, shea nut oil, soybean oil, tamanu oil, walnut oil, wheat germ oil, and the like.

In embodiments described herein, sunflower oil is present from about 2 wt. % to about 20 wt. % of the composition, coconut oil is present from about 2 wt. % to about 20 wt. % of the composition, shea butter is present from about 2 wt. % to about 20 wt. % of the composition, grapeseed oil is present from about 0.1 wt. % to about 10 wt. % of the composition, squalene is present from about 0.1 wt. % to about 10 wt. % of the composition, and tocopherol is present from about 0.1 wt. % to about 5 wt. % of the composition.

In embodiments described herein, sunflower oil is present from about 2 wt. % to about 20 wt. % of the composition, coconut oil is present from about 2 wt. % to about 20 wt. % of the composition, shea butter is present from about 2 wt. % to about 20 wt. % of the composition, camellia oil is present from about 1 wt. % to about 20 wt. % of the composition, squalane is present from about 0.1 wt. % to about 10 wt. % of the composition, cetyl alcohol is present from about 0.1 wt. % to about 10 wt. %, and tocopherol is present from about 0.1 wt. % to about 5 wt. % of the composition.

In embodiments described herein, sunflower oil is present from about 2 wt. % to about 20 wt. % of the composition, coconut oil is present from about 2 wt. % to about 20 wt. % of the composition, shea butter is present from about 2 wt. % to about 20 wt. % of the composition, camellia oil is present from about 1 wt. % to about 20 wt. % of the composition, squalane is present from about 0.1 wt. % to about 10 wt. % of the composition, cetyl alcohol is present from about 0.1 wt. % to about 10 wt. %, menthol is present from about 0.1 wt. % to about 5 wt. %, and tocopherol is present from about 0.1 wt. % to about 5 wt. % of the composition.

In embodiments described herein, sunflower oil is present from about 2 wt. % to about 20 wt. % of the composition, coconut oil is present from about 2 wt. % to about 20 wt. % of the composition, shea butter is present from about 2 wt. % to about 20 wt. % of the composition, camellia oil is present from about 1 wt. % to about 20 wt. % of the composition, squalane is present from about 0.1 wt. % to about 10 wt. % of the composition, cetyl alcohol is present from about 0.1 wt. % to about 10 wt. %, hemp oil is present from about 0.1 wt. % to about 20 wt. %, and tocopherol is present from about 0.1 wt. % to about 5 wt. % of the composition.

In embodiments described herein, sunflower oil is present from about 2 wt. % to about 20 wt. % of the composition, coconut oil is present from about 2 wt. % to about 20 wt. % of the composition, shea butter is present from about 2 wt. % to about 20 wt. % of the composition, camellia oil is present from about 1 wt. % to about 20 wt. % of the composition, squalane is present from about 0.1 wt. % to about 10 wt. % of the composition, cetyl alcohol is present from about 0.1 wt. % to about 10 wt. %, cannabidiol is present from about 0.1 wt. % to about 20 wt. %, and tocopherol is present from about 0.1 wt. % to about 5 wt. % of the composition.

In embodiments described herein, sunflower oil is present from about 2 wt. % to about 20 wt. % of the composition, coconut oil is present from about 2 wt. % to about 20 wt. % of the composition, shea butter is present from about 2 wt. % to about 20 wt. % of the composition, camellia oil is present from about 1 wt. % to about 20 wt. % of the composition, squalane is present from about 0.1 wt. % to about 10 wt. % of the composition, cetyl alcohol is present from about 0.1 wt. % to about 10 wt. %, menthol is present from about 0.1 wt. % to about 5 wt. %, hemp oil is present from about 0.1 wt. % to about 20 wt. %, and tocopherol is present from about 0.1 wt. % to about 5 wt. % of the composition.

In embodiments described herein, sunflower oil is present from about 2 wt. % to about 20 wt. % of the composition, coconut oil is present from about 2 wt. % to about 20 wt. % of the composition, shea butter is present from about 2 wt. % to about 20 wt. % of the composition, camellia oil is present from about 1 wt. % to about 20 wt. % of the composition, squalane is present from about 0.1 wt. % to about 10 wt. % of the composition, cetyl alcohol is present from about 0.1 wt. % to about 10 wt. %, menthol is present from about 0.1 wt. % to about 5 wt. %, cannabidiol is present from about 0.1 wt. % to about 20 wt. %, and tocopherol is present from about 0.1 wt. % to about 5 wt. % of the composition.

In embodiments described herein, sunflower oil is present from about 2 wt. % to about 10 wt. % of the composition, coconut oil is present from about 2 wt. % to about 10 wt. % of the composition, shea butter is present from about 2 wt. % to about 10 wt. % of the composition, grapeseed oil is present from about 0.1 wt. % to about 5 wt. % of the composition, squalene is present from about 0.1 wt. % to about 5 wt. % of the composition, and tocopherol is present from about 0.1 wt. % to about 2.5 wt. % of the composition.

In embodiments described herein, sunflower oil is present from about 2 wt. % to about 10 wt. % of the composition, coconut oil is present from about 2 wt. % to about 10 wt. % of the composition, shea butter is present from about 2 wt. % to about 10 wt. % of the composition, camellia oil is present from about 2 wt. % to about 10 wt. % of the composition, squalane is present from about 0.1 wt. % to about 7 wt. % of the composition, cetyl alcohol is present from about 1 wt. % to about 10 wt. %, and tocopherol is present from about 0.1 wt. % to about 2.5 wt. % of the composition.

In embodiments described herein, sunflower oil is present from about 2 wt. % to about 10 wt. % of the composition, coconut oil is present from about 2 wt. % to about 10 wt. % of the composition, shea butter is present from about 2 wt. % to about 10 wt. % of the composition, camellia oil is present from about 2 wt. % to about 10 wt. % of the composition, squalane is present from about 0.1 wt. % to about 7 wt. % of the composition, cetyl alcohol is present from about 1 wt. % to about 10 wt. %, menthol is present from about 0.1 wt. % to about 1 wt. %, and tocopherol is present from about 0.1 wt. % to about 2.5 wt. % of the composition.

In embodiments described herein, sunflower oil is present from about 2 wt. % to about 10 wt. % of the composition, coconut oil is present from about 2 wt. % to about 10 wt. % of the composition, shea butter is present from about 2 wt. % to about 10 wt. % of the composition, camellia oil is present from about 2 wt. % to about 10 wt. % of the composition, squalane is present from about 0.1 wt. % to about 7 wt. % of the composition, cetyl alcohol is present from about 1 wt. % to about 10 wt. %, hemp oil is present from about 0.1 wt. % to about 10 wt. %, and tocopherol is present from about 0.1 wt. % to about 2.5 wt. % of the composition.

In embodiments described herein, sunflower oil is present from about 2 wt. % to about 10 wt. % of the composition, coconut oil is present from about 2 wt. % to about 10 wt. % of the composition, shea butter is present from about 2 wt. % to about 10 wt. % of the composition, camellia oil is present from about 2 wt. % to about 10 wt. % of the composition, squalane is present from about 0.1 wt. % to about 7 wt. % of the composition, cetyl alcohol is present from about 1 wt. % to about 10 wt. %, cannabidiol is present from about 0.1 wt. % to about 10 wt. %, and tocopherol is present from about 0.1 wt. % to about 2.5 wt. % of the composition.

In embodiments described herein, sunflower oil is present from about 2 wt. % to about 10 wt. % of the composition, coconut oil is present from about 2 wt. % to about 10 wt. % of the composition, shea butter is present from about 2 wt. % to about 10 wt. % of the composition, camellia oil is present from about 2 wt. % to about 10 wt. % of the composition, squalane is present from about 0.1 wt. % to about 7 wt. % of the composition, cetyl alcohol is present from about 1 wt. % to about 10 wt. %, menthol is present from about 0.1 wt. % to about 1 wt. %, hemp oil is present from about 0.1 wt. % to about 10 wt. %, and tocopherol is present from about 0.1 wt. % to about 2.5 wt. % of the composition.

In embodiments described herein, sunflower oil is present from about 2 wt. % to about 10 wt. % of the composition, coconut oil is present from about 2 wt. % to about 10 wt. % of the composition, shea butter is present from about 2 wt. % to about 10 wt. % of the composition, camellia oil is present from about 2 wt. % to about 10 wt. % of the composition, squalane is present from about 0.1 wt. % to about 7 wt. % of the composition, cetyl alcohol is present from about 1 wt. % to about 10 wt. %, menthol is present from about 0.1 wt. % to about 1 wt. %, cannabidiol is present from about 0.1 wt. % to about 10 wt. %, and tocopherol is present from about 0.1 wt. % to about 2.5 wt. % of the composition.

In embodiments described herein, sunflower oil is present from about 2 wt. % to about 8 wt. % of the composition, coconut oil is present from about 2 wt. % to about 10 wt. % of the composition, shea butter is present from about 2 wt. % to about 5 wt. % of the composition, grapeseed oil is present from about 0.1 wt. % to about 3 wt. % of the composition, squalene is present from about 0.1 wt. % to about 3 wt. % of the composition, and tocopherol is present from about 0.1 wt. % to about 1.5 wt. % of the composition.

In embodiments described herein, sunflower oil is present from about 2 wt. % to about 8 wt. % of the composition, coconut oil is present from about 2 wt. % to about 10 wt. % of the composition, shea butter is present from about 2 wt. % to about 5 wt. % of the composition, camellia oil is present from about 5 wt. % to about 10 wt. % of the composition, squalane is present from about 1 wt. % to about 7 wt. % of the composition, cetyl alcohol is present from about 1 wt. % to about 5 wt. %, and tocopherol is present from about 0.1 wt. % to about 1.5 wt. % of the composition.

In embodiments described herein, sunflower oil is present from about 2 wt. % to about 8 wt. % of the composition, coconut oil is present from about 2 wt. % to about 10 wt. % of the composition, shea butter is present from about 2 wt. % to about 5 wt. % of the composition, camellia oil is present from about 5 wt. % to about 10 wt. % of the composition, squalane is present from about 1 wt. % to about 7 wt. % of the composition, cetyl alcohol is present from about 1 wt. % to about 5 wt. %, menthol is present from about 0.1 wt. % to about 0.5 wt. %, and tocopherol is present from about 0.1 wt. % to about 1.5 wt. % of the composition.

In embodiments described herein, sunflower oil is present from about 2 wt. % to about 8 wt. % of the composition, coconut oil is present from about 2 wt. % to about 10 wt. % of the composition, shea butter is present from about 2 wt. % to about 5 wt. % of the composition, camellia oil is present from about 5 wt. % to about 10 wt. % of the composition, squalane is present from about 1 wt. % to about 7 wt. % of the composition, cetyl alcohol is present from about 1 wt. % to about 5 wt. %, hemp oil is present from about 0.1 wt. % to about 5 wt. %, and tocopherol is present from about 0.1 wt. % to about 1.5 wt. % of the composition.

In embodiments described herein, sunflower oil is present from about 2 wt. % to about 8 wt. % of the composition, coconut oil is present from about 2 wt. % to about 10 wt. % of the composition, shea butter is present from about 2 wt. % to about 5 wt. % of the composition, camellia oil is present from about 5 wt. % to about 10 wt. % of the composition, squalane is present from about 1 wt. % to about 7 wt. % of the composition, cetyl alcohol is present from about 1 wt. % to about 5 wt. %, cannabidiol is present from about 0.1 wt. % to about 5 wt. %, and tocopherol is present from about 0.1 wt. % to about 1.5 wt. % of the composition.

In embodiments described herein, sunflower oil is present from about 2 wt. % to about 8 wt. % of the composition, coconut oil is present from about 2 wt. % to about 10 wt. % of the composition, shea butter is present from about 2 wt. % to about 5 wt. % of the composition, camellia oil is present from about 5 wt. % to about 10 wt. % of the composition, squalane is present from about 1 wt. % to about 7 wt. % of the composition, cetyl alcohol is present from about 1 wt. % to about 5 wt. %, menthol is present from about 0.1 wt. % to about 0.5 wt. %, hemp oil is present from about 0.1 wt. % to about 5 wt. %, and tocopherol is present from about 0.1 wt. % to about 1.5 wt. % of the composition.

In embodiments described herein, sunflower oil is present from about 2 wt. % to about 8 wt. % of the composition, coconut oil is present from about 2 wt. % to about 10 wt. % of the composition, shea butter is present from about 2 wt. % to about 5 wt. % of the composition, camellia oil is present from about 5 wt. % to about 10 wt. % of the composition, squalane is present from about 1 wt. % to about 7 wt. % of the composition, cetyl alcohol is present from about 1 wt. % to about 5 wt. %, menthol is present from about 0.1 wt. % to about 0.5 wt. %, cannabidiol is present from about 0.1 wt. % to about 5 wt. %, and tocopherol is present from about 0.1 wt. % to about 1.5 wt. % of the composition.

In embodiments described herein, sunflower oil is present at about 8 wt. % of the composition, coconut oil is present at about 10 wt. % of the composition, shea butter is present at about 5 wt. % of the composition, grapeseed oil is present at about 3 wt. % of the composition, squalene is present at about 3 wt. % of the composition, and tocopherol is present at about 1.5 wt. % of the composition.

In embodiments described herein, sunflower oil is present at about 5 wt. % of the composition, coconut oil is present at about 5 wt. % of the composition, shea butter is present at about 3 wt. % of the composition, camellia oil is present at about 7 wt. % of the composition, squalane is present at about 5 wt. % of the composition, and tocopherol is present at about 1 wt. % of the composition.

In embodiments described herein, sunflower oil is present at about 7 wt. % of the composition, coconut oil is present at about 5 wt. % of the composition, shea butter is present at about 2 wt. % of the composition, camellia oil is present at about 7 wt. % of the composition, squalane is present at about 6 wt. % of the composition, and tocopherol is present at about 1 wt. % of the composition.

In embodiments described herein, sunflower oil is present at about 8 wt. % of the composition, coconut oil is present at about 5 wt. % of the composition, shea butter is present at about 3 wt. % of the composition, camellia oil is present at about 9 wt. % of the composition, squalane is present at about 6 wt. % of the composition, and tocopherol is present at about 1 wt. % of the composition.

In embodiments described herein, sunflower oil is present at about 8 wt. % of the composition, coconut oil is present at about 5 wt. % of the composition, shea butter is present at about 3 wt. % of the composition, camellia oil is present at about 9 wt. % of the composition, squalane is present at about 6 wt. % of the composition, cetyl alcohol is present at about 2.5 wt. % of the composition, and tocopherol is present at about 1 wt. % of the composition.

In embodiments described herein, sunflower oil is present at about 8 wt. % of the composition, coconut oil is present at about 5 wt. % of the composition, shea butter is present at about 3 wt. % of the composition, camellia oil is present at about 9 wt. % of the composition, squalane is present at about 6 wt. % of the composition, menthol is present at about 0.2 wt. % of the composition, and tocopherol is present at about 1 wt. % of the composition.

In embodiments described herein, sunflower oil is present at about 8 wt. % of the composition, coconut oil is present at about 5 wt. % of the composition, shea butter is present at about 3 wt. % of the composition, camellia oil is present at about 9 wt. % of the composition, squalane is present at about 6 wt. % of the composition, hemp oil is present at about 0.2 wt. % of the composition, and tocopherol is present at about 1 wt. % of the composition.

In embodiments described herein, sunflower oil is present at about 8 wt. % of the composition, coconut oil is present at about 5 wt. % of the composition, shea butter is present at about 3 wt. % of the composition, camellia oil is present at about 9 wt. % of the composition, squalane is present at about 6 wt. % of the composition, cannabidiol is present at about 0.2 wt. % of the composition, and tocopherol is present at about 1 wt. % of the composition.

In embodiments described herein, sunflower oil is present at about 8 wt. % of the composition, coconut oil is present at about 5 wt. % of the composition, shea butter is present at about 3 wt. % of the composition, camellia oil is present at about 9 wt. % of the composition, squalane is present at about 6 wt. % of the composition, menthol is present at about 0.2 wt. % of the composition, hemp oil is present at about 0.2 wt. % of the composition, and tocopherol is present at about 1 wt. % of the composition.

In embodiments described herein, sunflower oil is present at about 8 wt. % of the composition, coconut oil is present at about 5 wt. % of the composition, shea butter is present at about 3 wt. % of the composition, camellia oil is present at about 9 wt. % of the composition, squalane is present at about 6 wt. % of the composition, menthol is present at about 0.2 wt. % of the composition, cannabidiol is present at about 0.2 wt. % of the composition, and tocopherol is present at about 1 wt. % of the composition.

In embodiments, the composition may further include a solvent, a chelating agent, an emulsifier, a thickener, a preservative, a fragrance, a buffer, a humectant, a surfactant, a diluent, a disintegrant, a filler, a binder, an emollient, a pigment, a cooling agent, and the like.

In embodiments, a solvent is selected from but not limited to water, ethanol, isopropanol, butanol, benzyl alcohol, ethylene glycol, propylene glycol, butanediols and isomers thereof, glycerol, glycerol triacetate, pentaerythritol, sorbitol, mannitol, Transcutol®, dimethyl isosorbide, polyethylene glycol, polypropylene glycol, polyvinylalcohol, hydroxypropyl methylcellulose, 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide, polyvinylpyrrolidone, ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof, and mixtures thereof. In embodiments, the solvent may be present from about 5 wt. % to about 90 wt. %, about 5 wt. % to about 80 wt. %, about 5 wt. % to about 70 wt. %, about 5 wt. % to about 60 wt. %, about 5 wt. % to about 50 wt. %, about 5 wt. % to about 40 wt. %, about 5 wt. % to about 30 wt. %, about 5 wt. % to about 20 wt. %, or about 5 wt. % to about 10 wt. % of the composition. Specific examples include about 5 wt. %, about 10 wt. %, about 25 wt. %, about 50 wt. %, about 58.2 wt. %, about 70 wt. %, about 90 wt. %, and any individual amount or any ranges between any two of these values.

In embodiments, emulsifiers can be selected from the group, but are not limited to, ceteth-20, laureth-3, glyceryl stearate, polyethylene glycol, macrogol cetostearyl ether, stearic acid, stearyl alcohol, polysorbate 60, Irish moss, Tween 80, sorbitol monostearate.glycol esters, fatty acids, fatty alcohols, fatty acid glycol esters, fatty esters, fatty ethers, esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanol amine cetyl phosphate, polysorbate 60, glyceryl stearate, PEG-100 stearate, tragacanth gum, 10-30 alkyl acrylate crosspolymers, and mixtures thereof. In embodiments, commercially available emulsifying wax may also be used. A typical emulsifying wax contains mixture of cetearyl alcohol, polysorbate 60, PEG-150 Stearate, Steareth-20, cetyl alcohol, and glyceryl stearate. In embodiments, the emulsifier may be present from about 1 wt. % to about 50 wt. %, about 1 wt. % to about 40 wt. %, about 1 wt. % to about 30 wt. %, about 1 wt. % to about 20 wt. %, about 1 wt. % to about 10 wt. %, or about 1 wt. % to about 5 wt. % of the composition. Specific examples include about 1 wt. %, about 2 wt. %, about 2.5 wt. %, about 4.5 wt. %, about 10 wt. %, about 25 wt. %, about 30 wt. %, about 40 wt. %, about 50 wt. %, and any individual amount or any ranges between any two of these values.

In embodiments, a chelating agent can be selected from the group, but are not limited to, tetrasodium 3-hydroxy-2,2′-iminodisuccinate (HIDS), ethylenediamine-N,N′-disuccinic acid (EDDS), iminodisuccinic acid, tetrasodium glutamate diacetate, nitrilotriacetic acid (NTA)), ethylenediamine tetraacetate (EDTA), and methylglycin diacetic acid (MGDA). In embodiments, the chelating agent may be present from about 0.01 wt. % to about 5 wt. %, about 0.01 wt. % to about 4 wt. %, about 0.01 wt. % to about 3 wt. %, about 0.01 wt. % to about 2 wt. %, about 0.01 wt. % to about 1 wt. %, or about 0.01 wt. % to about 0.5 wt. % of the composition. Specific examples include about 0.01 wt. %, about 0.1 wt. %, about 0.4 wt. %, about 0.5 wt. %, about 1 wt. %, about 5 wt. %, and any individual amount or any ranges between any two of these values.

In embodiments, a thickener can be selected from the group, but are not limited to, PEG-150 distearate, PEG-7 glyceryl cocoate, PEG-200 hydrogenated glyceryl palmitate, PEG-120 methyl glucose dioleate, carboxymethylene polymer, carboxyvinyl polymer, acrylates, C10-C30 alkyl acrylate crosspolymers, isopropyl myristate, and combinations thereof. Other examples include xantham gum, acacia, alginic acid, bentonite, carbopols, carbomer 940, carbomer 941, gelatin, carbomer copolymer, aluminum monostearat, dextrin, magnesium aluminum silicate, silicon dioxide, sodium alginate, triethanolamine, polyvinyl alcohol, pectin, methylcellulose, hydroxypropyl cellulose, aqueous thickening agents such as neutral, anionic-cationic polymers, and mixtures thereof. In embodiments, the thickener may be present from about 0.01 wt. % to about 5 wt. %, about 0.01 wt. % to about 4 wt. %, about 0.01 wt. % to about 3 wt. %, about 0.01 wt. % to about 2 wt. %, about 0.01 wt. % to about 1 wt. %, or about 0.01 wt. % to about 0.5 wt. % of the composition. Specific examples include about 0.01 wt. %, about 0.1 wt. %, about 0.4 wt. %, about 0.5 wt. %, about 1 wt. %, about 5 wt. %, and any individual amount or any ranges between any two of these values.

In embodiments, preservatives can be selected from the group, but not limited to, methylparaben and propylparaben and the salts thereof (e.g. sodium or potassium salts), potassium sorbate, sodium benzoate, diazolidinyi urea, phenoxyethanol, DMDM hydantoin, sorbic acid, benzyl alcohol, formaldehyde, triclosan.methylisothiazolinone, methylchloroisothiazolinone, caffeine, citric acid, benzoic acid, butylated hydroxytoluene, propylene glycol, organic acids, esters of parahydroxybenzoic acid (methyl, ethyl, propyl and butyl esters of parahydroxy benzoic acid, and their sodium salts etc), chloroform, chlorocresol, phenoxyethanol, quaternary ammonium compounds and butylated hydroxyanisole, and the mixtures thereof. In embodiments, the preservative may be present from about 0.01 wt. % to about 5 wt. %, about 0.01 wt. % to about 4 wt. %, about 0.01 wt. % to about 3 wt. %, about 0.01 wt. % to about 2 wt. %, about 0.01 wt. % to about 1 wt. %, or about 0.01 wt. % to about 0.5 wt. % of the composition. Specific examples include about 0.01 wt. %, about 0.1 wt. %, about 0.4 wt. %, about 0.5 wt. %, about 1 wt. %, about 5 wt. %, and any individual amount or any ranges between any two of these values.

In embodiments, a fragrance, such as aromatic agents can be selected from the group, but are not limited to, menthol, lavender, mint, cinnamon, chocolate, vanillin, and fruit extracts such as cherry, orange, strawberry, and grape, fennel oil, eucalyptus oil, carnation oil, ginger oil, sweet orange oil, tea tree oil, peppermint oil, and the mixtures thereof. In embodiments, the aromatic agents may be present from about 0.01 wt. % to about 5 wt. %, about 0.01 wt. % to about 4 wt. %, about 0.01 wt. % to about 3 wt. %, about 0.01 wt. % to about 2 wt. %, about 0.01 wt. % to about 1 wt. %, or about 0.01 wt. % to about 0.5 wt. % of the composition. Specific examples include about 0.01 wt. %, about 0.1 wt. %, about 0.5 wt. %, about 1 wt. %, about 5 wt. %, and any individual amount or any ranges between any two of these values.

In embodiments, the cooling agent is selected from but not limited to menthol; an isomer of menthol, a menthol derivative; 4-Methyl-3-(1-pyrrolidinyl)-2[5H]-furanone; WS-23, Icilin, Icilin Unilever Analog, 5-methyl-4-(1-pyrrolidinyl)-3-[2H]-furanone; 4,5-dimethyl-3-(1-pyrrolidinyl)-2 [5H]-furanone; isopulegol, 3-(1-menthoxy)propane-1,2-diol, 3-(1-menthoxy)-2-methylpropane-1,2-diol, p-menthane-2,3-diol, p-menthane-3,8-diol, 6-isopropyl-9-methyl-1,4-dioxas-piro [4,5]decane-2-methanol, menthyl succinate and its alkaline earth metal salts, trimethylcyclohexanol, N-ethyl-2-isopropyl-5-methylcyclohexanecarb-oxamide, Japanese mint (Mentha arvensis) oil, peppermint oil, menthone, menthone glycerol ketal, menthyl lactate, 3-(1-menthoxy)ethan-1-ol, 3-(1-menthoxy)propan-1-ol, 3-(1-menthoxy)butan-1-ol, 1-menthylacetic acid N-ethylamide, 1-menthyl-4-hydroxypentanoate, 1-menthyl-3-hydroxybutyrate, N,2,3-trimethyl-2-(1-methylethyl)-butanamide, and spearmint oil.

Emollient or lubricating vehicles that help hydrate the skin can also be used. In embodiments, emollients can be selected from the group, but are not limited to, liquid vaseline paraffinum liquidum, petrolatum, proplylene glycol, fatty acid esters, mineral oil including dimethicone, waxes including white wax, spermacetic wax, squalene, cetearyl alcohol, cetostearyl alcohol, stearyl alcohol, 2-Octyldodecanol, mineral oil USP, light mineral oil NF, liquid paraffin BP, light liquid paraffin BP. candellilla wax, sweet almond oil, apricot oil, emu oil, argan oil, glycerin, coconut oil, grape seed oil, honey, lanolinand, and mixtures thereof.

In embodiments, surfactants can be selected from the group, but are not limited to, a polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59; a polyoxyethylene alkylyl ether, polyoxyethylene cetyl ether, polyoxyethylene palmityl ether, polyethylene oxide hexadecyl ether, polyethylene glycol cetyl ether, a sucrose ester, a partial ester of sorbitol, sorbitan monolaurate, sorbitan monolaurate a monoglyceride, a diglyceride, isoceteth-20, a sucrose ester, or selected from the group consisting of steareth 2, glyceryl monostearate/PEG 100 stearate, Glyceryl Stearate, Steareth-21, peg 40 stearate, polysorbate 60, polysorbate 80, sorbitan stearate, laureth 4, Sorbitan monooleate, ceteareth 20, steareth 20, ceteth 20, Macrogol Cetostearyl Ether, ceteth 2, PEG-30 Dipolyhydroxystearate, sucrose distearate, polyoxyethylene (100) stearate, PEG 100 stearate, laureth 4, cetomacrogol ether, cetearyl alcohol, cetearyl glucoside, oleyl alcohol, steareth-2, diisopropyl adipate, capric/caprilic triglicerides, polysorbate 20, montanov 68 (cetearyl alcohol (and) cetearyl glucoside.), sharonmix 824 (a liquid blend of methyl paraben, ethyl paraben and propyl paraben—in phenoxyethanol), Simusol 165 (glyceryl stearate and PEG-100 stearate), methyl glucose sequistearate, Peg 30 dipolyhydroxystearate, sucrose stearic acid esters, sorbitan laureth, sorbitan stearate, and mixtures thereof.

In embodiments, buffer or pH adjusting agents can be selected from the group, but are not limited to, triethanolamine, triethylamine, diethylmethylamine, ethyldimethylamine, isopropyldimethylamine one or more adipic acids, glycines, citric acids, calcium hydroxides, magnesium aluminometasilicates, buffers, typically Bronsted-Lowry and/or Lewis acids and/or bases, or any combinations thereof.

In embodiments, the compositions may further include one or more additives selected from the group consisting of vitamins, cosmetic peptides, oil control agents, sensation modifying agents, skin lightening agents, hydrating compositions, a sunblock agent, a compound that absorbs or reflects UV photons, other skin care agent and combinations thereof.

In embodiments, the compositions may include other skin care agents, including, but not limited to, retinol, adapalene, steroids, sunblock, salicylate, minocycline, antifungals, peptides, antibodies, lidocaine, and the like and combinations thereof. In some embodiments, other skin care agents include N-acyl amino acid compounds including, for example, N-acyl phenylalanine, N-acyl tyrosine, and the like, their isomers, including their D and L isomers, salts, derivatives, and mixtures thereof. An example of a suitable N-acyl amino acid is N-undecylenoyl-L-phenylalanine is commercially available under the tradename SEPIWHITE®. Other skin active agents include, but are not limited to, Lavandox, Thallasine 2, Argireline NP, Gatuline In-Tense and Gatuline Expression, Myoxinol LS 9736, Syn-ake, and Instensyl®, Sesaflash™, N-acetyl D-glucosamine, panthenol (for example, DL panthenol available from Alps Pharmaceutical Inc.), tocopheryl nicotinate, benzoyl peroxide, 3-hydroxy benzoic acid, flavonoids (for example, flavanone, chalcone), farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinyl esters (for example, retinyl propionate), phytic acid, N-acetyl-L-cysteine, lipoic acid, tocopherol and its esters (for example, tocopheryl acetate: DL-a-tocopheryl acetate), azelaic acid, arachidonic acid, and others.

In embodiments, the compositions may include skin lightening agents, such as ascorbic acid compounds, vitamin B3 compounds, azelaic acid, butyl hydroxyanisole, gallic acid and its derivatives, glycyrrhizinic acid, hydroquinone, kojic acid, arbutin, mulberry extract, and combinations thereof.

In embodiments, the compositions may include sunblock agents, such as but not limited to para-aminobenzoic acid (PABA), PABA esters (glyceryl PABA, amyldimethyl PABA and octyldimethyl PABA), benzophenones (oxybenzone and sulisobenzone), cinnamates (octylmethoxy cinnamate and cinoxate), salicylates (homomethyl salicylate) anthranilates, TiO2, avobenzone, bemotrizinol, bisoctrizole, 3-(4-methylbenzylidene)-camphor, cinoxate, diethylamino hydroxybenzoyl hexyl benzoate, dioxybenzone, drometrizole trisiloxane, ecamsule, ethylhexyl triazone, homosalate, menthyl anthranilate, octocrylene, octyl salicylate, iscotrizinol, isopentenyl-4-methoxycinnamate, octyl-dimethyl-p-aminobenzoic acid, octyl-methoxycinnamate, oxybenzone, polysilicone-15, trolamine salicylate, and ZnO.

In embodiments, any of the compositions described herein may be a “morning regimen composition,” an “evening regimen composition,” or a “cooling composition.”

In embodiments, any of the compositions described herein may be a “morning regimen composition,” an “evening regimen composition,” or an “anti-itch regimen composition.”

In embodiments, any of the compositions described herein may be a “morning regimen composition,” an “evening regimen composition,” an “after wash regimen composition,” or a “fissure repair regimen composition.”

In embodiments, any of the compositions described herein may be a “daytime regimen composition” or an “evening regimen composition.”

In embodiments, any of the compositions described herein may be a “daytime balm regimen composition” or an “evening balm regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, grapeseed oil present from about 0.1 wt. % to about 10 wt. % of the composition, squalene present from about 0.1 wt. % to about 10 wt. % of the composition, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be a “morning regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be a “morning regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. % of the composition, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be a “morning regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. % of the composition, menthol present from about 0.1 wt. % to about 10 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be a “morning regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. % of the composition, hemp oil present from about 0.1 wt. % to about 10 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be a “morning regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. % of the composition, cannabidiol present from about 0.1 wt. % to about 10 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be a “morning regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. % of the composition, menthol present from about 0.1 wt. % to about 10 wt. %, hemp oil is present from about 0.1 wt. % to about 10 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be a “morning regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. % of the composition, menthol present from about 0.1 wt. % to about 10 wt. %, cannabidiol is present from about 0.1 wt. % to about 10 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be a “morning regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, grapeseed oil present from about 0.1 wt. % to about 10 wt. % of the composition, squalene present from about 0.1 wt. % to about 10 wt. % of the composition, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be an “evening regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be an “evening regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be an “evening regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. %, menthol present from about 0.1 wt. % to about 10 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be an “evening regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. %, hemp oil present from about 0.1 wt. % to about 10 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be an “evening regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. %, cannabidiol present from about 0.1 wt. % to about 10 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be an “evening regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. %, menthol present from about 0.1 wt. % to about 10 wt. %, hemp oil present from about 0.1 wt. % to about 10 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be an “evening regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. %, menthol present from about 0.1 wt. % to about 10 wt. %, cannabidiol present from about 0.1 wt. % to about 10 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be an “evening regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be an “anti-itch regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be an “anti-itch regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. %, menthol present from about 0.1 wt. % to about 10 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be an “anti-itch regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. %, hemp oil present from about 0.1 wt. % to about 10 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be an “anti-itch regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. %, cannabidiol present from about 0.1 wt. % to about 10 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be an “anti-itch regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. %, menthol present from about 0.1 wt. % to about 10 wt. %, hemp oil present from about 0.1 wt. % to about 10 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be an “anti-itch regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. %, menthol present from about 0.1 wt. % to about 10 wt. %, cannabidiol present from about 0.1 wt. % to about 10 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be an “anti-itch regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be an “after-wash regimen composition.”

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 1 wt. % to about 20 wt. % of the composition, squalane present from about 0.1 wt. % to about 10 wt. % of the composition, cetyl alcohol present from about 1 wt. % to about 20 wt. %, and tocopherol present from about 0.1 wt. % to about 5 wt. % of the composition. The composition may further include a solvent, a chelating agent, an emulsifier, a thickener, a preservative, and a fragrance, as disclosed herein. In embodiments, the composition may be an “after-wash regimen composition.”

In embodiments, the composition comprises natural oils present from about 0.1 wt. % to about 20 wt. % of the composition. The composition may further include a solvent, a chelating agent, an emulsifier, a thickener, a preservative, and a fragrance, as disclosed herein. In embodiments, the composition may be a “daytime balm regimen composition.”

In embodiments, the composition comprises natural oils present from about 0.1 wt. % to about 20 wt. % of the composition, and hemp oil present from about 0.1 wt. % to about 20 wt. % of the composition. The composition may further include a solvent, a chelating agent, an emulsifier, a thickener, a preservative, and a fragrance, as disclosed herein. In embodiments, the composition may be a “evening balm regimen composition.”

In embodiments, the composition comprises natural oils present from about 0.1 wt. % to about 20 wt. % of the composition, and cannabidiol present from about 0.1 wt. % to about 20 wt. % of the composition. The composition may further include a solvent, a chelating agent, an emulsifier, a thickener, a preservative, and a fragrance, as disclosed herein. In embodiments, the composition may be a “evening balm regimen composition.”

In embodiments, the morning regimen composition is less viscous when compared to the evening regimen composition.

In embodiments, the morning regimen composition is a thin cream that that is thicker than a lotion.

In embodiments, the evening regimen composition is a thick cream.

In embodiments, the composition comprises sunflower oil present from about 2 wt. % to about 20 wt. % of the composition, coconut oil present from about 2 wt. % to about 20 wt. % of the composition, shea butter present from about 2 wt. % to about 20 wt. % of the composition, grapeseed oil present from about 0.1 wt. % to about 10 wt. % of the composition, squalene present from about 0.1 wt. % to about 10 wt. % of the composition, tocopherol present from about 0.1 wt. % to about 5 wt. % of the composition, and a cooling agent present from about 0.1 wt. % to about 5 wt. % of the composition. The composition may further include a solvent, a chelating agent, an emulsifier, a thickener, a preservative, and a fragrance, as disclosed herein. In embodiments, the composition may be a “cooling composition.”

In embodiments, the compositions described herein may be formulated as a liquid. Liquid dosage forms for topical administration may include diluents such as, for example, alcohols, glycols, oils, water, and the like. Such compositions may also include wetting agents or emulsifiers. In embodiments, the compositions may be formulated as oil-in-water or water-in-oil emulsion. A cream can be a water-in-oil (w/o) emulsion in which an aqueous phase is dispersed in an oil phase, or an oil-in-water (o/w) emulsion in which an oil is dispersed within an aqueous base. An ointment generally refers to a more viscous oil-in-water cream. Traditional ointment bases (i.e. carrier) include hydrocarbons (petrolatum, beeswax, etc.) vegetable oils, fatty alcohols (cholesterol, lanolin, wool alcohol, stearyl alcohol, etc.) or silicones. Insoluble solids such as starch, zinc oxide, calcium carbonate, or talc can also be used in ointments and creams Gel forms of the compositions described above can be formed by the entrapment of large amounts of aqueous or aqueous-alcoholic liquids in a network of polymers or of colloidal solid particles. Such polymers or colloids (gelling or thickening agents) are typically present at concentrations of less than 10% w/w and include carboxymethyl cellulose, hydroxypropylmethyl cellulose, hydroxyethyl cellulose, methyl cellulose, sodium alginate, alginic acid, pectin, tragacanth, carrageen, agar, clays, aluminum silicate, carbomers, and the like.

In embodiments, the compositions may be formulated in any formulation suitable for topical administration, including, but not limited to, a solution, fluid, emulsion, suspension, solid, semi-solid, jelly, paste, gel, hydrogel, ointment, lotion, emulsion, cream, foam, mousse, liquid, spray, suspension, dispersion, powder, aerosol, or transdermal patch.

In embodiments, the compositions described above can be formulated as aerosols in which the composition is dissolved in a propellant such as dichlorodifluoromethane, trichlorofluoromethane, dichlorotetrafluoroethane, carbon dioxide, or other suitable gas, and a co-solvent such ethanol, acetone, hexadecyl alcohol, and the like and combinations thereof.

Pre-packaged kits may be provided containing the compositions described herein. In embodiments, the kit includes a plurality of separate containers, each containing a separate composition. The kit contains a first container containing a morning regimen composition, and a second container containing an evening regimen composition. In embodiments, the kit may also contain a third container containing a cooling composition that includes a cooling agent. The containers of the kit may be enclosed within a common outer packaging, such as, for example a cardboard or plastic box or a shrink wrap outer skin enclosing the various containers. The kits may be in the form of a consumer package or prescription package which provides the products described above. In embodiments, a combination of a consumer package with prescription product(s) may be obtained. The package may provide instructions or directions on how to use and/or combine the products for one or more treatment regimens as described above.

A wide variety of methods may be used for preparing the formulations described herein. Broadly speaking, the formulations may be prepared by combining together the components of the formulation, as described herein, at a temperature and for a time sufficient to provide a pharmaceutically acceptable composition. For example, in some embodiments, the compositions components of the compositions may be dissolved, suspended, dispersed or otherwise mixed in a selected carrier or vehicle, at an effective concentration such that the condition to be treated is relieved or ameliorated.

### Methods of Using Compositions Described Herein

Embodiments of the invention are directed to methods of ameliorating a skin condition in a subject in need thereof comprising topically administering to the subject a composition comprising sunflower oil, coconut oil, shea butter, grapeseed oil, squalene, and a tocopherol. In embodiments, the skin condition may be selected from eczema, erythema, irritation, pruritus, drying, drying of the skin, cracking of the skin, wrinkles, rosacea, contact dermatitis, atopic dermatitis, and combination thereof.

Embodiments of the invention are directed to methods of ameliorating a skin condition in a subject in need thereof comprising topically administering to the subject a composition comprising sunflower oil, coconut oil, shea butter, camellia oil, squalane, and a tocopherol. In embodiments, the skin condition may be selected from eczema, erythema, irritation, pruritus, drying, drying of the skin, cracking of the skin, wrinkles, rosacea, contact dermatitis, atopic dermatitis, and combination thereof.

Embodiments of the invention are directed to methods of ameliorating a skin condition in a subject in need thereof comprising topically administering to the subject a composition comprising sunflower oil, coconut oil, shea butter, camellia oil, squalane, cetyl alcohol, and a tocopherol. In embodiments, the skin condition may be selected from eczema, erythema, irritation, pruritus, drying, drying of the skin, cracking of the skin, wrinkles, rosacea, contact dermatitis, atopic dermatitis, and combination thereof.

Embodiments of the invention are directed to methods of ameliorating a skin condition in a subject in need thereof comprising topically administering to the subject a composition comprising sunflower oil, coconut oil, shea butter, camellia oil, squalane, cetyl alcohol, menthol, and a tocopherol. In embodiments, the skin condition may be selected from eczema, erythema, irritation, pruritus, drying, drying of the skin, cracking of the skin, wrinkles, rosacea, contact dermatitis, atopic dermatitis, and combination thereof.

Embodiments of the invention are directed to methods of ameliorating a skin condition in a subject in need thereof comprising topically administering to the subject a composition comprising sunflower oil, coconut oil, shea butter, camellia oil, squalane, cetyl alcohol, hemp oil, and a tocopherol. In embodiments, the skin condition may be selected from eczema, erythema, irritation, pruritus, drying, drying of the skin, cracking of the skin, wrinkles, rosacea, contact dermatitis, atopic dermatitis, and combination thereof.

Embodiments of the invention are directed to methods of ameliorating a skin condition in a subject in need thereof comprising topically administering to the subject a composition comprising sunflower oil, coconut oil, shea butter, camellia oil, squalane, cetyl alcohol, cannabidiol, and a tocopherol. In embodiments, the skin condition may be selected from eczema, erythema, irritation, pruritus, drying, drying of the skin, cracking of the skin, wrinkles, rosacea, contact dermatitis, atopic dermatitis, and combination thereof.

Embodiments of the invention are directed to methods of ameliorating a skin condition in a subject in need thereof comprising topically administering to the subject a composition comprising sunflower oil, coconut oil, shea butter, camellia oil, squalane, cetyl alcohol, menthol, hemp oil, and a tocopherol. In embodiments, the skin condition may be selected from eczema, erythema, irritation, pruritus, drying, drying of the skin, cracking of the skin, wrinkles, rosacea, contact dermatitis, atopic dermatitis, and combination thereof.

Embodiments of the invention are directed to methods of ameliorating a skin condition in a subject in need thereof comprising topically administering to the subject a composition comprising sunflower oil, coconut oil, shea butter, camellia oil, squalane, cetyl alcohol, menthol, cannabidiol, and a tocopherol. In embodiments, the skin condition may be selected from eczema, erythema, irritation, pruritus, drying, drying of the skin, cracking of the skin, wrinkles, rosacea, contact dermatitis, atopic dermatitis, and combination thereof.

Embodiments of the invention are directed to methods of ameliorating a skin condition in a subject in need thereof comprising topically administering to the subject a composition comprising natural oil. In embodiments, the skin condition may be selected from eczema, erythema, irritation, pruritus, drying, drying of the skin, cracking of the skin, wrinkles, rosacea, contact dermatitis, atopic dermatitis, and combination thereof.

Embodiments of the invention are directed to methods of ameliorating a skin condition in a subject in need thereof comprising topically administering to the subject a composition comprising natural oil and hemp oil. In embodiments, the skin condition may be selected from eczema, erythema, irritation, pruritus, drying, drying of the skin, cracking of the skin, wrinkles, rosacea, contact dermatitis, atopic dermatitis, and combination thereof.

Embodiments of the invention are directed to methods of ameliorating a skin condition in a subject in need thereof comprising topically administering to the subject a composition comprising natural oil and cannabidiol. In embodiments, the skin condition may be selected from eczema, erythema, irritation, pruritus, drying, drying of the skin, cracking of the skin, wrinkles, rosacea, contact dermatitis, atopic dermatitis, and combination thereof.

Embodiments of the invention are directed to methods of treating eczema in a subject in need thereof comprising topically administering to the subject a composition comprising sunflower oil, coconut oil, shea butter, grapeseed oil, squalene, and a tocopherol.

Embodiments of the invention are directed to methods of treating eczema in a subject in need thereof comprising topically administering to the subject a composition comprising sunflower oil, coconut oil, shea butter, camellia oil, squalane, and a tocopherol.

Embodiments of the invention are directed to methods of treating eczema in a subject in need thereof comprising topically administering to the subject a composition comprising sunflower oil, coconut oil, shea butter, camellia oil, squalane, cetyl alcohol, and a tocopherol.

Embodiments of the invention are directed to methods of treating eczema in a subject in need thereof comprising topically administering to the subject a composition comprising sunflower oil, coconut oil, shea butter, camellia oil, squalane, cetyl alcohol, menthol, and a tocopherol.

Embodiments of the invention are directed to methods of treating eczema in a subject in need thereof comprising topically administering to the subject a composition comprising sunflower oil, coconut oil, shea butter, camellia oil, squalane, cetyl alcohol, hemp oil, and a tocopherol.

Embodiments of the invention are directed to methods of treating eczema in a subject in need thereof comprising topically administering to the subject a composition comprising sunflower oil, coconut oil, shea butter, camellia oil, squalane, cetyl alcohol, cannabidiol, and a tocopherol.

Embodiments of the invention are directed to methods of treating eczema in a subject in need thereof comprising topically administering to the subject a composition comprising sunflower oil, coconut oil, shea butter, camellia oil, squalane, cetyl alcohol, menthol, hemp oil, and a tocopherol.

Embodiments of the invention are directed to methods of treating eczema in a subject in need thereof comprising topically administering to the subject a composition comprising sunflower oil, coconut oil, shea butter, camellia oil, squalane, cetyl alcohol, menthol, cannabidiol, and a tocopherol.

Embodiments of the invention are directed to methods of treating eczema in a subject in need thereof comprising topically administering to the subject a composition comprising sunflower oil, coconut oil, shea butter, grapeseed oil, squalene, a tocopherol, and a cooling agent.

Embodiments of the invention are directed to methods of treating drying of the skin in a subject in need thereof comprising topically administering to the subject a composition comprising sunflower oil, coconut oil, shea butter, grapeseed oil, squalene, and a tocopherol.

Embodiments of the invention are directed to methods of treating drying of the skin in a subject in need thereof comprising topically administering to the subject a composition comprising sunflower oil, coconut oil, shea butter, camellia oil, squalane, and a tocopherol.

Embodiments of the invention are directed to methods of treating drying of the skin in a subject in need thereof comprising topically administering to the subject a composition comprising sunflower oil, coconut oil, shea butter, camellia oil, squalane, cetyl alcohol, and a tocopherol.

Embodiments of the invention are directed to methods of treating drying of the skin in a subject in need thereof comprising topically administering to the subject a composition comprising sunflower oil, coconut oil, shea butter, camellia oil, squalane, cetyl alcohol, menthol, and a tocopherol.

Embodiments of the invention are directed to methods of treating drying of the skin in a subject in need thereof comprising topically administering to the subject a composition comprising sunflower oil, coconut oil, shea butter, camellia oil, squalane, cetyl alcohol, hemp oil, and a tocopherol.

Embodiments of the invention are directed to methods of treating drying of the skin in a subject in need thereof comprising topically administering to the subject a composition comprising sunflower oil, coconut oil, shea butter, camellia oil, squalane, cetyl alcohol, cannabidiol, and a tocopherol.

Embodiments of the invention are directed to methods of treating drying of the skin in a subject in need thereof comprising topically administering to the subject a composition comprising sunflower oil, coconut oil, shea butter, camellia oil, squalane, cetyl alcohol, menthol, hemp oil, and a tocopherol.

Embodiments of the invention are directed to methods of treating drying of the skin in a subject in need thereof comprising topically administering to the subject a composition comprising sunflower oil, coconut oil, shea butter, camellia oil, squalane, cetyl alcohol, menthol, cannabidiol, and a tocopherol.

Embodiments of the invention are directed to methods of treating drying of the skin in a subject in need thereof comprising topically administering to the subject a composition comprising sunflower oil, coconut oil, shea butter, grapeseed oil, squalene, a tocopherol, and a cooling agent.

Embodiments of the invention are directed to methods of treating pruritus in a subject in need thereof comprising topically administering to the subject a composition comprising sunflower oil, coconut oil, shea butter, grapeseed oil, squalene, and a tocopherol.

Embodiments of the invention are directed to methods of treating pruritus in a subject in need thereof comprising topically administering to the subject a composition comprising sunflower oil, coconut oil, shea butter, camellia oil, squalane, and a tocopherol.

Embodiments of the invention are directed to methods of treating pruritus in a subject in need thereof comprising topically administering to the subject a composition comprising sunflower oil, coconut oil, shea butter, camellia oil, squalane, cetyl alcohol, and a tocopherol.

Embodiments of the invention are directed to methods of treating pruritus in a subject in need thereof comprising topically administering to the subject a composition comprising sunflower oil, coconut oil, shea butter, camellia oil, squalane, cetyl alcohol, menthol, and a tocopherol.

Embodiments of the invention are directed to methods of treating pruritus in a subject in need thereof comprising topically administering to the subject a composition comprising sunflower oil, coconut oil, shea butter, camellia oil, squalane, cetyl alcohol, hemp oil, and a tocopherol.

Embodiments of the invention are directed to methods of treating pruritus in a subject in need thereof comprising topically administering to the subject a composition comprising sunflower oil, coconut oil, shea butter, camellia oil, squalane, cetyl alcohol, cannabidiol, and a tocopherol.

Embodiments of the invention are directed to methods of treating pruritus in a subject in need thereof comprising topically administering to the subject a composition comprising sunflower oil, coconut oil, shea butter, camellia oil, squalane, cetyl alcohol, menthol, hemp oil, and a tocopherol.

Embodiments of the invention are directed to methods of treating pruritus in a subject in need thereof comprising topically administering to the subject a composition comprising sunflower oil, coconut oil, shea butter, camellia oil, squalane, cetyl alcohol, menthol, cannabidiol, and a tocopherol.

Embodiments of the invention are directed to methods of treating pruritus in a subject in need thereof comprising topically administering to the subject a composition comprising sunflower oil, coconut oil, shea butter, grapeseed oil, squalene, a tocopherol, and a cooling agent.

Embodiments of the invention are directed to methods of improving skin texture or appearance in a subject in need thereof comprising topically administering to the subject a composition comprising sunflower oil, coconut oil, shea butter, grapeseed oil, squalene, and a tocopherol.

Embodiments of the invention are directed to methods of improving skin texture or appearance in a subject in need thereof comprising topically administering to the subject a composition comprising sunflower oil, coconut oil, shea butter, camellia oil, squalane, and a tocopherol.

Embodiments of the invention are directed to methods of improving skin texture or appearance in a subject in need thereof comprising topically administering to the subject a composition comprising sunflower oil, coconut oil, shea butter, camellia oil, squalane, cetyl alcohol, and a tocopherol.

Embodiments of the invention are directed to methods of improving skin texture or appearance in a subject in need thereof comprising topically administering to the subject a composition comprising sunflower oil, coconut oil, shea butter, camellia oil, squalane, cetyl alcohol, menthol, and a tocopherol.

Embodiments of the invention are directed to methods of improving skin texture or appearance in a subject in need thereof comprising topically administering to the subject a composition comprising sunflower oil, coconut oil, shea butter, camellia oil, squalane, cetyl alcohol, hemp oil, and a tocopherol.

Embodiments of the invention are directed to methods of improving skin texture or appearance in a subject in need thereof comprising topically administering to the subject a composition comprising sunflower oil, coconut oil, shea butter, camellia oil, squalane, cetyl alcohol, cannabidiol, and a tocopherol.

Embodiments of the invention are directed to methods of improving skin texture or appearance in a subject in need thereof comprising topically administering to the subject a composition comprising sunflower oil, coconut oil, shea butter, camellia oil, squalane, cetyl alcohol, menthol, hemp oil, and a tocopherol.

Embodiments of the invention are directed to methods of improving skin texture or appearance in a subject in need thereof comprising topically administering to the subject a composition comprising sunflower oil, coconut oil, shea butter, camellia oil, squalane, cetyl alcohol, menthol, cannabidiol, and a tocopherol.

Embodiments of the invention are directed to methods of improving skin texture or appearance in a subject in need thereof comprising topically administering to the subject a composition comprising natural oil.

Embodiments of the invention are directed to methods of improving skin texture or appearance in a subject in need thereof comprising topically administering to the subject a composition comprising natural oil and hemp oil.

Embodiments of the invention are directed to methods of improving skin texture or appearance in a subject in need thereof comprising topically administering to the subject a composition comprising natural oil and cannabidiol.

Embodiments of the invention are directed to methods of improving the appearance of or treating eczema in a subject in need thereof comprising topically administering to the subject a morning regimen composition comprising sunflower oil, coconut oil, shea butter, grapeseed oil, squalene, and a tocopherol, and an evening regimen composition comprising sunflower oil, coconut oil, shea butter, grapeseed oil, squalene, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, and a tocopherol, and an evening regimen composition comprising sunflower oil, coconut oil, shea butter, camellia oil, squalane, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, and a tocopherol, and an evening regimen composition comprising sunflower oil, coconut oil, shea butter, camellia oil, squalane, cetyl alcohol, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, menthol, and a tocopherol, and an evening regimen composition comprising sunflower oil, coconut oil, shea butter, camellia oil, squalane, cetyl alcohol, menthol, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, hemp oil, and a tocopherol, and an evening regimen composition comprising sunflower oil, coconut oil, shea butter, camellia oil, squalane, cetyl alcohol, hemp oil, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, cannabidiol, and a tocopherol, and an evening regimen composition comprising sunflower oil, coconut oil, shea butter, camellia oil, squalane, cetyl alcohol, cannabidiol, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, menthol, hemp oil, and a tocopherol, and an evening regimen composition comprising sunflower oil, coconut oil, shea butter, camellia oil, squalane, cetyl alcohol, menthol, hemp oil, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, menthol, cannabidiol, and a tocopherol, and an evening regimen composition comprising sunflower oil, coconut oil, shea butter, camellia oil, squalane, cetyl alcohol, menthol, cannabidiol, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, and a tocopherol, an evening regimen composition comprising sunflower oil, coconut oil, shea butter, camellia oil, squalane, and a tocopherol, and an anti-itch regimen composition comprising sunflower oil, coconut oil, shea butter, camellia oil, squalane, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, and a tocopherol, an evening regimen composition comprising sunflower oil, coconut oil, shea butter, camellia oil, squalane, cetyl alcohol, and a tocopherol, and an anti-itch regimen composition comprising sunflower oil, coconut oil, shea butter, camellia oil, squalane, cetyl alcohol, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, menthol, and a tocopherol, an evening regimen composition comprising sunflower oil, coconut oil, shea butter, camellia oil, squalane, cetyl alcohol, menthol, and a tocopherol, and an anti-itch regimen composition comprising sunflower oil, coconut oil, shea butter, camellia oil, squalane, cetyl alcohol, menthol, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, hemp oil, and a tocopherol, an evening regimen composition comprising sunflower oil, coconut oil, shea butter, camellia oil, squalane, cetyl alcohol, hemp oil, and a tocopherol, and an anti-itch regimen composition comprising sunflower oil, coconut oil, shea butter, camellia oil, squalane, cetyl alcohol, hemp oil, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, cannabidiol, and a tocopherol, an evening regimen composition comprising sunflower oil, coconut oil, shea butter, camellia oil, squalane, cetyl alcohol, cannabidiol, and a tocopherol, and an anti-itch regimen composition comprising sunflower oil, coconut oil, shea butter, camellia oil, squalane, cetyl alcohol, cannabidiol, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, menthol, hemp oil, and a tocopherol, an evening regimen composition comprising sunflower oil, coconut oil, shea butter, camellia oil, squalane, cetyl alcohol, menthol, hemp oil, and a tocopherol, and an anti-itch regimen composition comprising sunflower oil, coconut oil, shea butter, camellia oil, squalane, cetyl alcohol, menthol, hemp oil, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, menthol, cannabidiol, and a tocopherol, an evening regimen composition comprising sunflower oil, coconut oil, shea butter, camellia oil, squalane, cetyl alcohol, menthol, cannabidiol, and a tocopherol, and an anti-itch regimen composition comprising sunflower oil, coconut oil, shea butter, camellia oil, squalane, cetyl alcohol, menthol, cannabidiol, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, and a tocopherol, an evening regimen composition comprising sunflower oil, coconut oil, shea butter, camellia oil, squalane, and a tocopherol, and an after-wash regimen composition comprising sunflower oil, coconut oil, shea butter, camellia oil, squalane, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, and a tocopherol, an evening regimen composition comprising sunflower oil, coconut oil, shea butter, camellia oil, squalane, cetyl alcohol, and a tocopherol, and an after-wash regimen composition comprising sunflower oil, coconut oil, shea butter, camellia oil, squalane, cetyl alcohol, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, menthol, and a tocopherol, an evening regimen composition comprising sunflower oil, coconut oil, shea butter, camellia oil, squalane, cetyl alcohol, menthol, and a tocopherol, and an after-wash regimen composition comprising sunflower oil, coconut oil, shea butter, camellia oil, squalane, cetyl alcohol, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, hemp oil, and a tocopherol, an evening regimen composition comprising sunflower oil, coconut oil, shea butter, camellia oil, squalane, cetyl alcohol, hemp oil, and a tocopherol, and an after-wash regimen composition comprising sunflower oil, coconut oil, shea butter, camellia oil, squalane, cetyl alcohol, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, cannabidiol, and a tocopherol, an evening regimen composition comprising sunflower oil, coconut oil, shea butter, camellia oil, squalane, cetyl alcohol, cannabidiol, and a tocopherol, and an after-wash regimen composition comprising sunflower oil, coconut oil, shea butter, camellia oil, squalane, cetyl alcohol, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, menthol, hemp oil, and a tocopherol, an evening regimen composition comprising sunflower oil, coconut oil, shea butter, camellia oil, squalane, cetyl alcohol, menthol, hemp oil, and a tocopherol, and an after-wash regimen composition comprising sunflower oil, coconut oil, shea butter, camellia oil, squalane, cetyl alcohol, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, menthol, cannabidiol, and a tocopherol, an evening regimen composition comprising sunflower oil, coconut oil, shea butter, camellia oil, squalane, cetyl alcohol, menthol, cannabidiol, and a tocopherol, and an after-wash regimen composition comprising sunflower oil, coconut oil, shea butter, camellia oil, squalane, cetyl alcohol, and a tocopherol.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, and a tocopherol, an evening regimen composition comprising sunflower oil, coconut oil, shea butter, camellia oil, squalane, and a tocopherol, an after-wash regimen composition comprising sunflower oil, coconut oil, shea butter, camellia oil, squalane, and a tocopherol, and a fissure repair regimen composition comprising natural oil and butter.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, and a tocopherol, an evening regimen composition comprising sunflower oil, coconut oil, shea butter, camellia oil, squalane, cetyl alcohol, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, menthol, and a tocopherol, an evening regimen composition comprising sunflower oil, coconut oil, shea butter, camellia oil, squalane, cetyl alcohol, menthol, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, hemp oil, and a tocopherol, an evening regimen composition comprising sunflower oil, coconut oil, shea butter, camellia oil, squalane, cetyl alcohol, hemp oil, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, cannabidiol, and a tocopherol, an evening regimen composition comprising sunflower oil, coconut oil, shea butter, camellia oil, squalane, cetyl alcohol, cannabidiol, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, menthol, hemp oil, and a tocopherol, an evening regimen composition comprising sunflower oil, coconut oil, shea butter, camellia oil, squalane, cetyl alcohol, menthol, hemp oil, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

Embodiments of the invention are directed to methods of improving the appearance of or treating eczema in a subject in need thereof comprising topically administering to the subject a morning regimen composition comprising sunflower oil, coconut oil, shea butter, camellia oil, squalane, cetyl alcohol, menthol, cannabidiol, and a tocopherol, an evening regimen composition comprising sunflower oil, coconut oil, shea butter, camellia oil, squalane, cetyl alcohol, menthol, cannabidiol, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

Embodiments of the invention are directed to methods of improving the appearance of or treating drying of the skin in a subject in need thereof comprising topically administering to the subject a morning regimen composition comprising sunflower oil, coconut oil, shea butter, grapeseed oil, squalene, and a tocopherol, and an evening regimen composition comprising sunflower oil, coconut oil, shea butter, grapeseed oil, squalene, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, and a tocopherol, and an evening regimen composition comprising sunflower oil, coconut oil, shea butter, camellia oil, squalane, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, and a tocopherol, and an evening regimen composition comprising sunflower oil, coconut oil, shea butter, camellia oil, squalane, cetyl alcohol, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, menthol, and a tocopherol, and an evening regimen composition comprising sunflower oil, coconut oil, shea butter, camellia oil, squalane, cetyl alcohol, menthol, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, hemp oil, and a tocopherol, and an evening regimen composition comprising sunflower oil, coconut oil, shea butter, camellia oil, squalane, cetyl alcohol, hemp oil, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, cannabidiol, and a tocopherol, and an evening regimen composition comprising sunflower oil, coconut oil, shea butter, camellia oil, squalane, cetyl alcohol, cannabidiol, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, menthol, hemp oil, and a tocopherol, and an evening regimen composition comprising sunflower oil, coconut oil, shea butter, camellia oil, squalane, cetyl alcohol, menthol, hemp oil, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, menthol, cannabidiol, and a tocopherol, and an evening regimen composition comprising sunflower oil, coconut oil, shea butter, camellia oil, squalane, cetyl alcohol, menthol, cannabidiol, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, and a tocopherol, an evening regimen composition comprising sunflower oil, coconut oil, shea butter, camellia oil, squalane, and a tocopherol, and an anti-itch regimen composition comprising sunflower oil, coconut oil, shea butter, camellia oil, squalane, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, and a tocopherol, an evening regimen composition comprising sunflower oil, coconut oil, shea butter, camellia oil, squalane, cetyl alcohol, and a tocopherol, and an anti-itch regimen composition comprising sunflower oil, coconut oil, shea butter, camellia oil, squalane, cetyl alcohol, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, menthol, and a tocopherol, an evening regimen composition comprising sunflower oil, coconut oil, shea butter, camellia oil, squalane, cetyl alcohol, menthol, and a tocopherol, and an anti-itch regimen composition comprising sunflower oil, coconut oil, shea butter, camellia oil, squalane, cetyl alcohol, menthol, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, hemp oil, and a tocopherol, an evening regimen composition comprising sunflower oil, coconut oil, shea butter, camellia oil, squalane, cetyl alcohol, hemp oil, and a tocopherol, and an anti-itch regimen composition comprising sunflower oil, coconut oil, shea butter, camellia oil, squalane, cetyl alcohol, hemp oil, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, cannabidiol, and a tocopherol, an evening regimen composition comprising sunflower oil, coconut oil, shea butter, camellia oil, squalane, cetyl alcohol, cannabidiol, and a tocopherol, and an anti-itch regimen composition comprising sunflower oil, coconut oil, shea butter, camellia oil, squalane, cetyl alcohol, cannabidiol, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, menthol, hemp oil, and a tocopherol, an evening regimen composition comprising sunflower oil, coconut oil, shea butter, camellia oil, squalane, cetyl alcohol, menthol, hemp oil, and a tocopherol, and an anti-itch regimen composition comprising sunflower oil, coconut oil, shea butter, camellia oil, squalane, cetyl alcohol, menthol, hemp oil, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, menthol, cannabidiol, and a tocopherol, an evening regimen composition comprising sunflower oil, coconut oil, shea butter, camellia oil, squalane, cetyl alcohol, menthol, cannabidiol, and a tocopherol, and an anti-itch regimen composition comprising sunflower oil, coconut oil, shea butter, camellia oil, squalane, cetyl alcohol, menthol, cannabidiol, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, and a tocopherol, an evening regimen composition comprising sunflower oil, coconut oil, shea butter, camellia oil, squalane, and a tocopherol, and an after-wash regimen composition comprising sunflower oil, coconut oil, shea butter, camellia oil, squalane, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, and a tocopherol, an evening regimen composition comprising sunflower oil, coconut oil, shea butter, camellia oil, squalane, cetyl alcohol, and a tocopherol, and an after-wash regimen composition comprising sunflower oil, coconut oil, shea butter, camellia oil, squalane, cetyl alcohol, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, menthol, and a tocopherol, an evening regimen composition comprising sunflower oil, coconut oil, shea butter, camellia oil, squalane, cetyl alcohol, menthol, and a tocopherol, and an after-wash regimen composition comprising sunflower oil, coconut oil, shea butter, camellia oil, squalane, cetyl alcohol, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, hemp oil, and a tocopherol, an evening regimen composition comprising sunflower oil, coconut oil, shea butter, camellia oil, squalane, cetyl alcohol, hemp oil, and a tocopherol, and an after-wash regimen composition comprising sunflower oil, coconut oil, shea butter, camellia oil, squalane, cetyl alcohol, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, cannabidiol, and a tocopherol, an evening regimen composition comprising sunflower oil, coconut oil, shea butter, camellia oil, squalane, cetyl alcohol, cannabidiol, and a tocopherol, and an after-wash regimen composition comprising sunflower oil, coconut oil, shea butter, camellia oil, squalane, cetyl alcohol, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, menthol, hemp oil, and a tocopherol, an evening regimen composition comprising sunflower oil, coconut oil, shea butter, camellia oil, squalane, cetyl alcohol, menthol, hemp oil, and a tocopherol, and an after-wash regimen composition comprising sunflower oil, coconut oil, shea butter, camellia oil, squalane, cetyl alcohol, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, menthol, cannabidiol, and a tocopherol, an evening regimen composition comprising sunflower oil, coconut oil, shea butter, camellia oil, squalane, cetyl alcohol, menthol, cannabidiol, and a tocopherol, and an after-wash regimen composition comprising sunflower oil, coconut oil, shea butter, camellia oil, squalane, cetyl alcohol, and a tocopherol.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, and a tocopherol, an evening regimen composition comprising sunflower oil, coconut oil, shea butter, camellia oil, squalane, and a tocopherol, an after-wash regimen composition comprising sunflower oil, coconut oil, shea butter, camellia oil, squalane, and a tocopherol, and a fissure repair regimen composition comprising natural oil and butter.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, and a tocopherol, an evening regimen composition comprising sunflower oil, coconut oil, shea butter, camellia oil, squalane, cetyl alcohol, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, menthol, and a tocopherol, an evening regimen composition comprising sunflower oil, coconut oil, shea butter, camellia oil, squalane, cetyl alcohol, menthol, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, hemp oil, and a tocopherol, an evening regimen composition comprising sunflower oil, coconut oil, shea butter, camellia oil, squalane, cetyl alcohol, hemp oil, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, cannabidiol, and a tocopherol, an evening regimen composition comprising sunflower oil, coconut oil, shea butter, camellia oil, squalane, cetyl alcohol, cannabidiol, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, menthol, hemp oil, and a tocopherol, an evening regimen composition comprising sunflower oil, coconut oil, shea butter, camellia oil, squalane, cetyl alcohol, menthol, hemp oil, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

Embodiments of the invention are directed to methods of improving the appearance of or treating drying of the skin in a subject in need thereof comprising topically administering to the subject a morning regimen composition comprising sunflower oil, coconut oil, shea butter, camellia oil, squalane, cetyl alcohol, menthol, cannabidiol, and a tocopherol, an evening regimen composition comprising sunflower oil, coconut oil, shea butter, camellia oil, squalane, cetyl alcohol, menthol, cannabidiol, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

Embodiments of the invention are directed to methods of improving or treating pruritus in a subject in need thereof comprising topically administering to the subject a morning regimen composition comprising sunflower oil, coconut oil, shea butter, grapeseed oil, squalene, and a tocopherol, and an evening regimen composition comprising sunflower oil, coconut oil, shea butter, grapeseed oil, squalene, and a tocopherol.

Embodiments of the invention are directed to methods of improving the appearance of or treating pruritus in a subject in need thereof comprising topically administering to the subject a morning regimen composition comprising sunflower oil, coconut oil, shea butter, camellia oil, squalane, and a tocopherol, and an evening regimen composition comprising sunflower oil, coconut oil, shea butter, camellia oil, squalane, and a tocopherol.

Embodiments of the invention are directed to methods of improving the appearance of or treating pruritus in a subject in need thereof comprising topically administering to the subject a morning regimen composition comprising sunflower oil, coconut oil, shea butter, camellia oil, squalane, cetyl alcohol, and a tocopherol, and an evening regimen composition comprising sunflower oil, coconut oil, shea butter, camellia oil, squalane, cetyl alcohol, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, menthol, and a tocopherol, and an evening regimen composition comprising sunflower oil, coconut oil, shea butter, camellia oil, squalane, cetyl alcohol, menthol, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, hemp oil, and a tocopherol, and an evening regimen composition comprising sunflower oil, coconut oil, shea butter, camellia oil, squalane, cetyl alcohol, hemp oil, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, cannabidiol, and a tocopherol, and an evening regimen composition comprising sunflower oil, coconut oil, shea butter, camellia oil, squalane, cetyl alcohol, cannabidiol, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, menthol, hemp oil, and a tocopherol, and an evening regimen composition comprising sunflower oil, coconut oil, shea butter, camellia oil, squalane, cetyl alcohol, menthol, hemp oil, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, menthol, cannabidiol, and a tocopherol, and an evening regimen composition comprising sunflower oil, coconut oil, shea butter, camellia oil, squalane, cetyl alcohol, menthol, cannabidiol, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, and a tocopherol, an evening regimen composition comprising sunflower oil, coconut oil, shea butter, camellia oil, squalane, and a tocopherol, and an anti-itch regimen composition comprising sunflower oil, coconut oil, shea butter, camellia oil, squalane, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, and a tocopherol, an evening regimen composition comprising sunflower oil, coconut oil, shea butter, camellia oil, squalane, cetyl alcohol, and a tocopherol, and an anti-itch regimen composition comprising sunflower oil, coconut oil, shea butter, camellia oil, squalane, cetyl alcohol, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, menthol, and a tocopherol, an evening regimen composition comprising sunflower oil, coconut oil, shea butter, camellia oil, squalane, cetyl alcohol, menthol, and a tocopherol, and an anti-itch regimen composition comprising sunflower oil, coconut oil, shea butter, camellia oil, squalane, cetyl alcohol, menthol, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, hemp oil, and a tocopherol, an evening regimen composition comprising sunflower oil, coconut oil, shea butter, camellia oil, squalane, cetyl alcohol, hemp oil, and a tocopherol, and an anti-itch regimen composition comprising sunflower oil, coconut oil, shea butter, camellia oil, squalane, cetyl alcohol, hemp oil, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, cannabidiol, and a tocopherol, an evening regimen composition comprising sunflower oil, coconut oil, shea butter, camellia oil, squalane, cetyl alcohol, cannabidiol, and a tocopherol, and an anti-itch regimen composition comprising sunflower oil, coconut oil, shea butter, camellia oil, squalane, cetyl alcohol, cannabidiol, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, menthol, hemp oil, and a tocopherol, an evening regimen composition comprising sunflower oil, coconut oil, shea butter, camellia oil, squalane, cetyl alcohol, menthol, hemp oil, and a tocopherol, and an anti-itch regimen composition comprising sunflower oil, coconut oil, shea butter, camellia oil, squalane, cetyl alcohol, menthol, hemp oil, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, menthol, cannabidiol, and a tocopherol, an evening regimen composition comprising sunflower oil, coconut oil, shea butter, camellia oil, squalane, cetyl alcohol, menthol, cannabidiol, and a tocopherol, and an anti-itch regimen composition comprising sunflower oil, coconut oil, shea butter, camellia oil, squalane, cetyl alcohol, menthol, cannabidiol, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, and a tocopherol, an evening regimen composition comprising sunflower oil, coconut oil, shea butter, camellia oil, squalane, and a tocopherol, and an after-wash regimen composition comprising sunflower oil, coconut oil, shea butter, camellia oil, squalane, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, and a tocopherol, an evening regimen composition comprising sunflower oil, coconut oil, shea butter, camellia oil, squalane, cetyl alcohol, and a tocopherol, and an after-wash regimen composition comprising sunflower oil, coconut oil, shea butter, camellia oil, squalane, cetyl alcohol, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, menthol, and a tocopherol, an evening regimen composition comprising sunflower oil, coconut oil, shea butter, camellia oil, squalane, cetyl alcohol, menthol, and a tocopherol, and an after-wash regimen composition comprising sunflower oil, coconut oil, shea butter, camellia oil, squalane, cetyl alcohol, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, hemp oil, and a tocopherol, an evening regimen composition comprising sunflower oil, coconut oil, shea butter, camellia oil, squalane, cetyl alcohol, hemp oil, and a tocopherol, and an after-wash regimen composition comprising sunflower oil, coconut oil, shea butter, camellia oil, squalane, cetyl alcohol, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, cannabidiol, and a tocopherol, an evening regimen composition comprising sunflower oil, coconut oil, shea butter, camellia oil, squalane, cetyl alcohol, cannabidiol, and a tocopherol, and an after-wash regimen composition comprising sunflower oil, coconut oil, shea butter, camellia oil, squalane, cetyl alcohol, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, menthol, hemp oil, and a tocopherol, an evening regimen composition comprising sunflower oil, coconut oil, shea butter, camellia oil, squalane, cetyl alcohol, menthol, hemp oil, and a tocopherol, and an after-wash regimen composition comprising sunflower oil, coconut oil, shea butter, camellia oil, squalane, cetyl alcohol, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, menthol, cannabidiol, and a tocopherol, an evening regimen composition comprising sunflower oil, coconut oil, shea butter, camellia oil, squalane, cetyl alcohol, menthol, cannabidiol, and a tocopherol, and an after-wash regimen composition comprising sunflower oil, coconut oil, shea butter, camellia oil, squalane, cetyl alcohol, and a tocopherol.

Embodiments of the invention are directed to methods of improving or treating pruritus in a subject in need thereof comprising topically administering to the subject a morning regimen composition comprising sunflower oil, coconut oil, shea butter, camellia oil, squalane, and a tocopherol, an evening regimen composition comprising sunflower oil, coconut oil, shea butter, camellia oil, squalane, and a tocopherol, an after-wash regimen composition comprising sunflower oil, coconut oil, shea butter, camellia oil, squalane, and a tocopherol, and a fissure repair regimen composition comprising natural oil and butter.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, and a tocopherol, an evening regimen composition comprising sunflower oil, coconut oil, shea butter, camellia oil, squalane, cetyl alcohol, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, menthol, and a tocopherol, an evening regimen composition comprising sunflower oil, coconut oil, shea butter, camellia oil, squalane, cetyl alcohol, menthol, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, hemp oil, and a tocopherol, an evening regimen composition comprising sunflower oil, coconut oil, shea butter, camellia oil, squalane, cetyl alcohol, hemp oil, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, cannabidiol, and a tocopherol, an evening regimen composition comprising sunflower oil, coconut oil, shea butter, camellia oil, squalane, cetyl alcohol, cannabidiol, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, menthol, hemp oil, and a tocopherol, an evening regimen composition comprising sunflower oil, coconut oil, shea butter, camellia oil, squalane, cetyl alcohol, menthol, hemp oil, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

Embodiments of the invention are directed to methods of improving or treating pruritus in a subject in need thereof comprising topically administering to the subject a morning regimen composition comprising sunflower oil, coconut oil, shea butter, camellia oil, squalane, cetyl alcohol, menthol, cannabidiol, and a tocopherol, an evening regimen composition comprising sunflower oil, coconut oil, shea butter, camellia oil, squalane, cetyl alcohol, menthol, cannabidiol, and a tocopherol, an after-wash regimen composition comprising sunflower oil, coconut oil, shea butter, camellia oil, squalane, cetyl alcohol, and a tocopherol, and a fissure repair regimen composition comprising natural oil and butter.

In embodiments, the methods also comprise administration by transdermal delivery, transcutaneous delivery, percutaneous delivery, or transmucosal delivery of the compositions disclosed herein.

In some embodiments, a morning regimen composition is applied once in the morning, followed by a gap of 12 hours, and an evening regimen composition is applied once in the evening. In some embodiments, the composition may be administered as needed, once daily, once in two days, once in three days, twice a week, every other week, every other day, or the like for one or more dosing cycles. A dosing cycle may include administration for about 1 week, about 2 weeks, about 3 weeks, about 4 weeks, about 5 weeks, about 6 weeks, about 7 weeks, about 8 weeks, about 9 weeks, or about 10 weeks.

In some embodiments, a morning regimen composition is applied once in the morning, followed by a gap of 12 hours, and an evening regimen composition is applied once in the evening. In some embodiments, an anti-itch regimen composition is applied prior to or after application of the morning regimen composition. In some embodiments, the anti-itch regimen composition is applied prior to or after application of the evening regimen composition. In some embodiments, the composition may be administered as needed, once daily, once in two days, once in three days, twice a week, every other week, every other day, or the like for one or more dosing cycles. A dosing cycle may include administration for about 1 week, about 2 weeks, about 3 weeks, about 4 weeks, about 5 weeks, about 6 weeks, about 7 weeks, about 8 weeks, about 9 weeks, or about 10 weeks.

In some embodiments, a morning regimen composition is applied once in the morning, followed by a gap of 12 hours, and an evening regimen composition is applied once in the evening. In some embodiments, an after washing regimen composition is applied prior to or after application of the morning regimen composition.

In some embodiments, the after washing regimen composition is applied prior to or after application of the evening regimen composition. In some embodiments, a fissure repair regimen composition is applied prior to or after application of the morning regimen composition. In some embodiments, the fissure repair regimen composition is applied prior to or after application of the evening regimen composition. In some embodiments, the composition may be administered as needed, once daily, once in two days, once in three days, twice a week, every other week, every other day, or the like for one or more dosing cycles. A dosing cycle may include administration for about 1 week, about 2 weeks, about 3 weeks, about 4 weeks, about 5 weeks, about 6 weeks, about 7 weeks, about 8 weeks, about 9 weeks, or about 10 weeks.

In some embodiments, a morning regimen composition is applied once in the morning, followed by a gap of 8-12 hours, and an evening regimen composition is applied once in the evening. In some embodiments, the composition may be administered as needed, once daily, once in two days, once in three days, twice a week, every other week, every other day, or the like for one or more dosing cycles. A dosing cycle may include administration for about 1 week, about 2 weeks, about 3 weeks, about 4 weeks, about 5 weeks, about 6 weeks, about 7 weeks, about 8 weeks, about 9 weeks, or about 10 weeks.

In some embodiments, a morning regimen composition is applied once in the morning, followed by a gap of 8-12 hours, and an evening regimen composition is applied once in the evening. In some embodiments, an anti-itch regimen composition is applied prior to or after application of the morning regimen composition. In some embodiments, the anti-itch regimen composition is applied prior to or after application of the evening regimen composition. In some embodiments, the composition may be administered as needed, once daily, once in two days, once in three days, twice a week, every other week, every other day, or the like for one or more dosing cycles. A dosing cycle may include administration for about 1 week, about 2 weeks, about 3 weeks, about 4 weeks, about 5 weeks, about 6 weeks, about 7 weeks, about 8 weeks, about 9 weeks, or about 10 weeks.

In some embodiments, a morning regimen composition is applied once in the morning, followed by a gap of 8-12 hours, and an evening regimen composition is applied once in the evening. In some embodiments, an after wash regimen composition is applied prior to or after application of the morning regimen composition. In some embodiments, the after wash regimen composition is applied prior to or after application of the evening regimen composition. In some embodiments, a fissure repair regimen composition is applied prior to or after application of the morning regimen composition. In some embodiments, the fissure repair regimen composition is applied prior to or after application of the evening regimen composition. In some embodiments, the composition may be administered as needed, once daily, once in two days, once in three days, twice a week, every other week, every other day, or the like for one or more dosing cycles. A dosing cycle may include administration for about 1 week, about 2 weeks, about 3 weeks, about 4 weeks, about 5 weeks, about 6 weeks, about 7 weeks, about 8 weeks, about 9 weeks, or about 10 weeks.

In some embodiments, only a morning regimen composition is administered. In some embodiments, only an evening regimen composition is administered. In some embodiments, the morning regimen composition is administered followed by administration of the evening regimen composition next day.

In some embodiments, only a morning regimen composition is administered. In some embodiments, only an evening regimen composition is administered. In some embodiments, only an anti-itch regimen composition is administered. In some embodiments, the morning regimen composition is administered followed by administration of the evening regimen composition next day. In some embodiments, the morning regimen composition and the anti-itch composition are administered followed by administration of the evening regimen composition next day.

In some embodiments, only a morning regimen composition is administered. In some embodiments, only an evening regimen composition is administered. In some embodiments, only an after wash regimen composition is administered. In some embodiments, only a fissure repair regimen composition is administered. In some embodiments, the morning regimen composition is administered followed by administration of the evening regimen composition next day. In some embodiments, the morning regimen composition and the after wash composition are administered followed by administration of the evening regimen composition next day. In some embodiments, the morning regimen composition and the fissure repair composition are administered followed by administration of the evening regimen composition next day.

In some embodiments, only a daytime balm regimen composition is administered. In some embodiments, only an evening balm regimen composition is administered. In some embodiments, the daytime balm regimen composition is administered followed by administration of the evening balm regimen composition.

In some embodiments, the composition comprising cooling agent can be administered as needed anytime.

In some embodiments, the methods may include a variety of additional steps including, for example, cleaning the surface tissue at the site of applying and the like.

In embodiments, the methods may further include ablation of the tissue surface before, during or after administration of the compositions described herein. In embodiments, tissue surface ablation may include electromagnetic radiation, laser, dermal abrasion, chemical peel, ultrasound, heating, cooling, or by a needle.

In embodiments, a starting kit is provided. In embodiments, the starting kit comprises a “morning regimen composition,” an “evening regimen composition,” and a “cooling composition.” The kit may further contain directions or instructions for applying the compositions. In embodiments, the directions or instructions for applying the compositions may comprise instructing the subject to topically apply the morning regimen composition once a day in the morning, topically apply the evening regimen composition once a day in the evening and topically apply the cooling composition as needed.

In embodiments, a starting kit is provided. In embodiments, the starting kit comprises a “morning regimen composition,” an “evening regimen composition,” and a “anti-itch composition.” The kit may further contain directions or instructions for applying the compositions. In embodiments, the directions or instructions for applying the compositions may comprise instructing the subject to topically apply the morning regimen composition once a day in the morning, and topically apply the evening regimen composition once a day in the evening as needed.

In embodiments, a starting kit is provided. In embodiments, the starting kit comprises a “morning regimen composition,” an “evening regimen composition,” an “after-wash composition,” and a “fissure repair composition.” The kit may further contain directions or instructions for applying the compositions. In embodiments, the directions or instructions for applying the compositions may comprise instructing the subject to topically apply the morning regimen composition once a day in the morning, and topically apply the evening regimen composition once a day in the evening as needed.

In embodiments, a starting kit is provided. In embodiments, the starting kit comprises a “daytime balm regimen composition” and an “evening balm regimen composition.” The kit may further contain directions or instructions for applying the compositions. In embodiments, the directions or instructions for applying the compositions may comprise instructing the subject to topically apply the daytime regimen composition once a day in the morning, topically apply the evening balm regimen composition once a day in the evening as needed.

In embodiments, a fissure repair regimen composition is provided in a container. In embodiments, the container is a collapsible tube. The collapsible tube may comprise a volume from about 0.1 ounce to about 5 ounces, from about 0.1 ounce to about 4 ounces, from about 0.1 ounce to about 3 ounces, from about 0.1 ounce to about 2 ounces, from about 0.1 ounce to about 1 ounces, from about 0.1 ounce to about 0.5 ounces. Specific examples include 0.1 ounce, 0.2 ounces, 0.3 ounces, 0.4 ounces, 0.5 ounces, 0.6 ounces, 0.7 ounces, 0.8 ounces, 0.9 ounces, 1 ounce, 2 ounces, 3 ounces, 4 ounces, 5 ounces. The collapsible tube may have dimensions of 0.75 inch diameter, 3.375 inch length, and a 0.045 inch orifice at a tip of the collapsible tube.

In embodiments, a daytime balm regimen composition is provided in a container. In embodiments, the container is a tube or a sphere. The container may comprise a volume from about 0.1 ounce to about 5 ounces, from about 0.1 ounce to about 4 ounces, from about 0.1 ounce to about 3 ounces, from about 0.1 ounce to about 2 ounces, from about 0.1 ounce to about 1 ounces, from about 0.1 ounce to about 0.5 ounces. Specific examples include 0.1 ounce, 0.2 ounces, 0.3 ounces, 0.4 ounces, 0.5 ounces, 0.6 ounces, 0.7 ounces, 0.8 ounces, 0.9 ounces, 1 ounce, 2 ounces, 3 ounces, 4 ounces, 5 ounces.

### EXAMPLES

### Example 1

An exemplary composition is described below:

| component | Wt % |
|---|---|
| water | 58.2 |
| Tetrasodium glutamate diacetate | 0.4 |
| Sunflower oil | 8 |
| Coconut oil | 10 |
| Emulsifying wax | 10 |
| Shea butter | 5 |
| Grapeseed oil | 3 |
| Squalene | 3 |
| Mixed tocopherol-T50 | 1.5 |
| Xantham gum | 0.4 |
| Potassium sorbate | 0.4 |
| Fragrance | 0.1 |

### Example 2

An exemplary composition is described below:

| component | Wt % |
|---|---|
| water | 62.2 |
| Camellia Oleifera Seed Oil | 7 |
| Sunflower oil | 5 |
| Coconut oil | 5 |
| Squalane | 5 |
| Cetyl Alcohol | 4.5 |
| Shea butter | 3 |
| Glyceryl Stearate | 2.25 |
| PEG-100 Stearate | 2.25 |
| Glycerin | 2 |
| Tocopherol | 1 |
| Xanthan Gum | 0.4 |
| Sodium Benzoate | 0.4 |

### Example 3

An exemplary composition is described below:

| component | Wt % |
|---|---|
| water | 61.2 |
| Camellia Oleifera Seed Oil | 7 |
| Sunflower oil | 7 |
| Squalane | 6 |
| Coconut oil | 5 |
| Glycerin | 4 |
| Glyceryl Stearate | 2 |
| PEG-100 Stearate | 2 |
| Shea butter | 2 |
| Cetyl Alcohol | 2 |
| Tocopherol | 1 |
| Xanthan Gum | 0.4 |
| Sodium Benzoate | 0.4 |

### Example 4

An exemplary composition is described below:

| component | Wt % |
|---|---|
| water | 57.3 |
| Camellia Oleifera Seed Oil | 9 |
| Sunflower oil | 8 |
| Squalane | 6 |
| Coconut oil | 5 |
| Shea butter | 3 |
| Glycerin | 3 |
| Cetyl Alcohol | 2.5 |
| Glyceryl Stearate | 2 |
| PEG-100 Stearate | 2 |
| Tocopherol | 1 |
| Xanthan Gum | 0.4 |
| Sodium Benzoate | 0.4 |
| Menthol | 0.2 |

### Example 5

An exemplary composition is described below:

| component | Wt % |
|---|---|
| water | 57.3 |
| Camellia Oleifera Seed Oil | 9 |
| Sunflower oil | 8 |
| Squalane | 6 |
| Coconut oil | 5 |
| Shea butter | 3 |
| Glycerin | 3 |
| Cetyl Alcohol | 2.5 |
| Glyceryl Stearate | 2 |
| PEG-100 Stearate | 2 |
| Tocopherol | 1 |
| Xanthan Gum | 0.4 |
| Sodium Benzoate | 0.4 |
| Hemp oil | 0.2 |

### Example 6

An exemplary composition is described below:

| component | Wt % |
|---|---|
| water | 57.3 |
| Camellia Oleifera Seed Oil | 9 |
| Sunflower oil | 8 |
| Squalane | 6 |
| Coconut oil | 5 |
| Shea butter | 3 |
| Glycerin | 3 |
| Cetyl Alcohol | 2.5 |
| Glyceryl Stearate | 2 |
| PEG-100 Stearate | 2 |
| Tocopherol | 1 |
| Xanthan Gum | 0.4 |
| Sodium Benzoate | 0.4 |
| Cannabidiol | 0.2 |

What is claimed is:

1. A topical composition comprising:
sunflower oil, coconut oil, shea butter, camellia oil, squalane, a tocopherol, and at least one selected from the group consisting of menthol, hemp oil, and cannabidiol.

2. The topical composition of claim 1, further comprising a solvent, a chelating agent, an emulsifier, a thickener, a preservative, and a fragrance.

3. The topical composition of claim 1, wherein
the sunflower oil is present from about 2 wt. % to about 20 wt. % of the composition,
the coconut oil is present from about 2 wt. % to about 20 wt. % of the composition,
the shea butter is present from about 2 wt. % to about 20 wt. % of the composition,
the camellia oil is present from about 1 wt. % to about 20 wt. % of the composition,
the squalane is present from about 1 wt. % to about 20 wt. % of the composition, and
the tocopherol is present from about 0.1 wt. % to about 5 wt. % of the composition.

4. The topical composition of claim 1, wherein
the menthol is present from about 0.1 wt. % to about 10 wt. % of the composition;
the hemp oil is present from about 0.1 wt. % to about 20 wt. % of the composition; and
the cannabidiol is present from about 0.1 wt. % to about 20 wt. % of the composition;

5. The topical composition of claim 2, wherein the solvent is selected from the group consisting of water, ethanol, isopropanol, butanol, benzyl alcohol, ethylene glycol, propylene glycol, butanediol, glycerol, glycerol triacetate, pentaerythritol, sorbitol, mannitol, dimethyl isosorbide, polyethylene glycol, polypropylene glycol, polyvinylalcohol, hydroxypropyl methylcellulose, and combinations thereof.

6. The topical composition of claim 2, wherein the chelating agent is selected from the group consisting of tetrasodium 3-hydroxy-2,2′-iminodisuccinate (HIDS), ethylenediamine-N,N′-disuccinic acid (EDDS), iminodisuccinic acid, tetrasodium glutamate diacetate, nitrilotriacetic acid (NTA)), ethylenediamine tetraacetate (EDTA), methylglycin diacetic acid (MGDA), and combinations thereof.

7. The topical composition of claim 2, wherein the emulsifier is selected from the group consisting of cetyl alcohol, emulsifying wax, ceteth-20, laureth-3, glyceryl stearate, polyethylene glycol, macrogol cetostearyl ether, stearic acid, stearyl alcohol, polysorbate 60, Irish moss, Tween 80, and combinations thereof.

8. The topical composition of claim 2, wherein the thickener is selected from the group consisting of include xantham gum, acacia, alginic acid, bentonite, carbopols, carbomer 940, carbomer 941, gelatin, carbomer copolymer, aluminum monostearat, dextrin, magnesium aluminum silicate, silicon dioxide, sodium alginate, triethanolamine, polyvinyl alcohol, pectin, methylcellulose, hydroxypropyl cellulose, and combinations thereof.

9. The topical composition of claim 2, wherein the preservative is selected from the group consisting of methylparaben, propylparaben, potassium sorbate, sodium benzoate, diazolidinyi urea, phenoxyethanol, DMDM hydantoin, sorbic acid, benzyl alcohol, formaldehyde, triclosan.methylisothiazolinone, methylchloroisothiazolinone, caffeine, citric acid, benzoic acid, butylated hydroxytoluene, propylene glycol, and combinations thereof.

10. The topical composition of claim 2, wherein the fragrance is selected from the group consisting of menthol, lavender, mint, cinnamon, chocolate, vanillin, cherry fruit extract, orange fruit extract, strawberry fruit extract, grape fruit extract, fennel oil, eucalyptus oil, carnation oil, ginger oil, sweet orange oil, peppermint oil, tea tree oil, and combinations thereof.

11. A method of ameliorating a skin condition in a subject comprising topically administering to the subject a composition comprising sunflower oil, coconut oil, shea butter, camellia oil, squalane, a tocopherol, and at least one selected from the group consisting of menthol, hemp oil, and cannabidiol.

12. The method of claim 11, wherein the skin condition is selected from eczema, erythema, irritation, pruritus, drying, drying of the skin, cracking of the skin, wrinkles, rosacea, contact dermatitis, atopic dermatitis, and combination thereof.

13. The method of claim 11, wherein the composition comprises sunflower oil from about 2 wt. % to about 20 wt. % of the composition, coconut oil from about 2 wt. % to about 20 wt. % of the composition, shea butter from about 2 wt. % to about 20 wt. % of the composition, camellia oil from about 0.1 wt. % to about 20 wt. % of the composition, squalane from about 1 wt. % to about 20 wt. % of the composition, and tocopherol from about 0.1 wt. % to about 5 wt. % of the composition.

14. The method of claim 11, wherein the composition comprises menthol from about 0.1 wt. % to about 10 wt. % of the composition.

15. A method of improving an appearance of eczema in a subject comprising topically administering to the subject:
a morning regimen composition comprising sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 0.1 wt. % to about 20 wt. % of the composition, squalane present from about 1 wt. % to about 20 wt. % of the composition, tocopherol present from about 0.1 wt. % to about 5 wt. % of the composition, and at least one selected from the group consisting of menthol present from about 0.1 wt. % to about 10 wt. %, hemp oil present from about 0.1 wt. % to about 20 wt. %, and cannabidiol present from about 0.1 wt. % to about 20 wt. %;
an evening regimen composition comprising sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 0.1 wt. % to about 20 wt. % of the composition, squalane present from about 1 wt. % to about 20 wt. % of the composition, tocopherol present from about 0.1 wt. % to about 5 wt. % of the composition, and at least one selected from the group consisting of menthol present from about 0.1 wt. % to about 10 wt. %, hemp oil present from about 0.1 wt. % to about 20 wt. %, and cannabidiol present from about 0.1 wt. % to about 20 wt. %; and
optionally administering to the subject as needed an anti-itch composition comprising sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 0.1 wt. % to about 20 wt. % of the composition, squalane present from about 1 wt. % to about 20 wt. % of the composition, tocopherol present from about 0.1 wt. % to about 5 wt. % of the composition, and at least one selected from the group consisting of menthol present from about 0.1 wt. % to about 10 wt. %, hemp oil present from about 0.1 wt. % to about 20 wt. %, and cannabidiol present from about 0.1 wt. % to about 20 wt. %.

16. The method of claim 15, further comprising administering to the subject as needed an after-wash composition comprising sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 0.1 wt. % to about 20 wt. % of the composition, squalane present from about 1 wt. % to about 20 wt. % of the composition, and a tocopherol present from about 0.1 wt. % to about 5 wt. % of the composition; and
optionally administering to the subject as needed a fissure repair composition comprising natural oil and butter.

17. A method of improving an appearance of drying of the skin in a subject comprising topically administering to the subject:
a morning regimen composition comprising sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 0.1 wt. % to about 20 wt. % of the composition, squalane present from about 1 wt. % to about 20 wt. % of the composition, tocopherol present from about 0.1 wt. % to about 5 wt. % of the composition, and at least one selected from the group consisting of menthol present from about 0.1 wt. % to about 10 wt. %, hemp oil present from about 0.1 wt. % to about 20 wt. %, and cannabidiol present from about 0.1 wt. % to about 20 wt. %;
an evening regimen composition comprising sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 0.1 wt. % to about 20 wt. % of the composition, squalane present from about 1 wt. % to about 20 wt. % of the composition, tocopherol present from about 0.1 wt. % to about 5 wt. % of the composition, and at least one selected from the group consisting of menthol present from about 0.1 wt. % to about 10 wt. %, hemp oil present from about 0.1 wt. % to about 20 wt. %, and cannabidiol present from about 0.1 wt. % to about 20 wt. %; and
optionally administering to the subject as needed an anti-itch composition comprising sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 0.1 wt. % to about 20 wt. % of the composition, squalane present from about 1 wt. % to about 20 wt. % of the composition, tocopherol present from about 0.1 wt. % to about 5 wt. % of the composition, and at least one selected from the group consisting of menthol present from about 0.1 wt. % to about 10 wt. %, hemp oil present from about 0.1 wt. % to about 20 wt. %, and cannabidiol present from about 0.1 wt. % to about 20 wt. %.

18. The method of claim 17, further comprising administering to the subject as needed an after-wash composition comprising sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 0.1 wt. % to about 20 wt. % of the composition, squalane present from about 1 wt. % to about 20 wt. % of the composition, and a tocopherol present from about 0.1 wt. % to about 5 wt. % of the composition; and
optionally administering to the subject as needed a fissure repair composition comprising natural oil and butter.

19. A method of improving an appearance of pruritus in a subject comprising topically administering to the subject:
a morning regimen composition comprising sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 0.1 wt. % to about 20 wt. % of the composition, squalane present from about 1 wt. % to about 20 wt. % of the composition, tocopherol present from about 0.1 wt. % to about 5 wt. % of the composition, and at least one selected from the group consisting of menthol present from about 0.1 wt. % to about 10 wt. %, hemp oil present from about 0.1 wt. % to about 20 wt. %, and cannabidiol present from about 0.1 wt. % to about 20 wt. %;
an evening regimen composition comprising sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 0.1 wt. % to about 20 wt. % of the composition, squalane present from about 1 wt. % to about 20 wt. % of the composition, tocopherol present from about 0.1 wt. % to about 5 wt. % of the composition, and at least one selected from the group consisting of menthol present from about 0.1 wt. % to about 10 wt. %, hemp oil present from about 0.1 wt. % to about 20 wt. %, and cannabidiol present from about 0.1 wt. % to about 20 wt. %; and
optionally administering to the subject as needed an anti-itch composition comprising sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 0.1 wt. % to about 20 wt. % of the composition, squalane present from about 1 wt. % to about 20 wt. % of the composition, tocopherol present from about 0.1 wt. % to about 5 wt. % of the composition, and at least one selected from the group consisting of menthol present from about 0.1 wt. % to about 10 wt. %, hemp oil present from about 0.1 wt. % to about 20 wt. %, and cannabidiol present from about 0.1 wt. % to about 20 wt. %.

20. The method of claim 19, further comprising administering to the subject as needed an after-wash composition comprising sunflower oil present from about 2 wt. % to about 20 wt. % of the composition, coconut oil present from about 2 wt. % to about 20 wt. % of the composition, shea butter present from about 2 wt. % to about 20 wt. % of the composition, camellia oil present from about 0.1 wt. % to about 20 wt. % of the composition, squalane present from about 1 wt. % to about 20 wt. % of the composition, and a tocopherol present from about 0.1 wt. % to about 5 wt. % of the composition; and
optionally administering to the subject as needed a fissure repair composition comprising natural oil and butter.

21. A kit comprising a morning regimen composition, an evening regimen composition, an after-wash composition, a fissure repair composition, and instructions for using the compositions.
